(19) United States
(12) Patent Application Publication
(10) Pub. No.: US 2025/0241560 A1
(43) Pub. Date: Jul. 31, 2025

(54) COMPOSITIONS AND METHODS FOR MEASURING OSMOLARITY

(71) Applicant: Arkansas State University – Jonesboro, State University, AR (US)

(72) Inventor: Anahita Izadyar, State University, AR (US)

(21) Appl. No.: 19/041,146

(22) Filed: Jan. 30, 2025

Related U.S. Application Data
(60) Provisional application No. 63/626,821, filed on Jan. 30, 2024.

(51) Int. Cl. A61B 5/145; A61B 5/1477; G01N 27/30; G01N 27/333

(52) U.S. Cl. CPC A61B 5/14507; A61B 5/14546; A61B 5/1477; G01N 27/307; G01N 27/308; G01N 27/3335

(57) ABSTRACT

The present invention is directed to an electrochemical sensor for the measurement of osmolarity.

This application claims priority to U.S. Provisional Application No. 63/626,821 filed on Jan. 30, 2024, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This disclosure relates to compositions and methods for measuring osmolarity.

### BACKGROUND OF THE INVENTION

Diagnosing Dry Eye Disease (DED) poses specific challenges. Current diagnostic methods—such as tear breakup time, Schirmer test, and tear osmolarity—have limitations, including subjective interpretation, result variability, and incomplete assessment of tear film composition.

### SUMMARY OF THE INVENTION

Aspects of the disclosure are drawn towards a screen-printed, solid contact ion-sensitive electrode (SP-SC-ISE), comprising one or more screen-printed electrodes, wherein at least one electrode is coated with a sensing membrane comprises a conducting layer and an ion-sensing membrane. In embodiments, the in the screen-printed electrode is selected from a carbon electrode or a glass carbon electrode. In embodiments, the conducting layer comprises one or more conductive polymers, nanoparticles, or composite thereof. In embodiments, the conductive polymer or composite thereof comprises poly (3,4-ethylene dioxythiophene) polystyrene sulfonate (PEDOT:PSS), graphene oxide (GO), PTh Polythiophene (PTh), and Polyacetylene (PA), or a combination thereof. In embodiments, the ion-sensing composite membrane comprises a polymer, a solvent mediator, an ionophore, and a solvent. In embodiments, the polymer comprises polyvinyl chloride (PVC). In embodiments, the ionophore comprises a Na⁺ ionophore. In embodiments, the Na⁺ ionophore comprises lipophilic sodium tetrakis (4-fluorophenyl) borate dehydrate Na-ionophore. In embodiments, the solvent comprises tetrahydrofuran (THF). In embodiments, the solvent mediator is selected from the group consisting of 2-nitrophenyl octyl ether (O-NPOE), Dioctylsebacate (DOS), di-2-ethylhexyl phthalate (DEHP), or any combination thereof. In embodiments, the ion-sensing composite membrane has less than about 10% cross-sensitivity to contaminants. In embodiments, the one or more screen-printed electrodes comprise a working electrode, a reference electrode, and a counter electrode. In embodiments, the conducting layer is deposited on the surface of the working electrode or on the surface of the working electrode, the reference electrode, and the counter electrode. In embodiments, the ion-sensing composite membrane is deposited on the conducting layer. In embodiments, the SP-SC-ISE is disposable. In embodiments, the SP-SC-ISE is paper-based.

Aspects of the disclosure are drawn towards an electrochemical sensor for measuring the osmolarity of a sample comprising one or more electrodes and a sensing membrane, wherein the sensing membrane comprises a conducting layer and an ion-sensing membrane. In embodiments, the one or more electrodes comprise a screen-printed electrode (SPE). In embodiments, the sensor is a voltametric sensor. In embodiments, the sensor is a microsensor. In embodiments, the sensor is disposable. In embodiments, the sensor is reusable. In embodiments, the microsensor comprises disposable tips. In embodiments, the sensor is paper based. In embodiments, the one or more electrodes is selected from a counter electrode, a working electrode, a reference electrode, or a combination thereof. In embodiments, the counter electrode comprises platinum. In embodiments, the working electrode is selected from a glass carbon electrode or a carbon electrode. In embodiments, reference electrode comprises silver/silver chloride (Ag/AgCl) or saturated calomel electrode (SCE). In embodiments, working electrode is coated with the sensing membrane described herein. In embodiments, the sensing membrane contacts the working electrode, the counter electrode, and the reference electrode. In embodiments, sensor can perform or be subjected to ion-transfer stripping voltammetry (ITSV). In embodiments, the conductive layer comprises one or more conductive polymers, nanoparticles, or composite thereof. In embodiments, the conductive polymer or composite thereof comprises poly (3,4-ethylene dioxythiophene) polystyrene sulfonate (PEDOT:PSS), graphene oxide (GO), PTh Polythiophene (PTh), and Polyacetylene (PA), or a combination thereof. In embodiments, the ion-sensing membrane comprises an ionophore composite. In embodiments, the ionophore composite comprises a polymer, a solvent mediator, an ionophore, and a solvent. In embodiments, the ionophore comprise a Na⁺ ionophore. In embodiments, the Na⁺ ionophore comprises lipophilic sodium tetrakis (4-fluorophenyl) borate dehydrate Na-ionophore. In embodiments, the polymer comprises polyvinyl chloride (PVC). In embodiments, the solvent comprises THF. In embodiments, the solvent mediator is selected from the group consisting of 2-nitrophenyl octyl ether (O-NPOE), Dioctylsebacate (DOS), di-2-ethylhexyl phthalate (DEHP), or any combination thereof. In embodiments, the sensor has a detection limit of about 0.005 mM. In embodiments, the sensor can measure a sample volume of about 1 μL to about 50 μL. In embodiments, sensor further comprises an anti-fouling agent. In embodiments, anti-fouling agent comprises a polar chemical group, hydrophilic polymers, self-assembled layers, or a combination thereof. In embodiments, the sensor comprises a microfluidic suction probe or flow-through sensor. In embodiments, the sensor can be in communication with another electrochemical device.

Aspects of the disclosure are drawn towards a method of diagnosing Dry Eye Disease (DED), the method comprising: applying a sensor described herein to the tears of a subject; measuring the osmolarity of the tears; and determining if the subject is afflicted with Dry Eye Disease (DED) based upon the osmolarity. In embodiments, the measuring osmolarity comprises measuring the physical or electrochemical signal of a target analyte in the sample, converting the sensor signal to osmolarity by measuring the sensor signal at known concentrations of a reference solution to generate a concentration curve, and multiplying the concentration of the sodium ion by the van't Hoff factor, 2.

Other objects and advantages of this invention will become readily apparent from the ensuing description.

### BRIEF DESCRIPTION OF THE FIGURES

FIG. 1 shows non-limiting, exemplary data. Panel (A) OCP using SC-SP-ISE, in concentration range of 3.37×10⁻⁵ to 3.37×10⁻¹ M of Nat. Panel (B) Calibration Plot (R²=0.9923), Nernstian slope of 56.6.

FIG. 2 shows a non-limiting, exemplary schematic illustration of a gold microelectrode.

FIG. 3 shows non-limiting, exemplary ion-transfer stripping voltammetry (ITSV) data. Panel (A) Stripping voltammograms of sodium ion, 7 min preconcentration at 0.05 V. Scan rate 0.05 V/s. Panel (B) Calibration plots of background-subtracted peak current versus of sodium ion concentration (circles) and best fit (solid line).

FIG. 4 shows a non-limiting, exemplary schematic of cation transfer by ion transfer stripping voltammetry with PVC membranes coated on a conductive polymer modified solid electrode.

FIG. 5 shows a non-limiting, exemplary schematic of the preparation of microelectrodes with a laser puller.

FIG. 6 shows a non-limiting, exemplary schematics of a microsensor described herein. Panel a shows a non-limiting exemplary schematic of a microsensor comprising 3 electrodes. Panel b shows a non-limiting, exemplary illustration of an embodiment of a microsensor described herein. In embodiments, the microsensor can be placed in a tip designed to suction the solution toward the microelectrode. For example, the tip can be a probe for diagnostic or monitoring purposes.

FIG. 7 shows a non-limiting, exemplary schematic of the effect of high osmolarity on the cornea.

FIG. 8 shows a non-limiting, exemplary schematic of the overview of solution contact with SC-ISEs to detect Na⁺.

FIG. 9 shows a non-limiting, exemplary Schematic Representation of the modified SPE based on PVC composite and CPs/nanomaterials.

FIG. 10 shows a non-limiting, exemplary schematic of tear osmolarity levels.

FIG. 11 shows a non-limiting, exemplary illustration of a screen-printed electrode as described herein. In this non-limiting illustration, three electrodes-working, reference, and counter-are integrated into a single compact unit. The sensor can have all the electrodes pre-arranged for use without needing to assemble multiple components. The illustration indicates that the sensor can be in communication with an electrochemical microfluidic device.

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Electrochemical Sensor

Aspects of the invention are drawn towards an electrochemical sensor and a sensor system for electrochemically detecting and/or measuring at least one analyte in a fluid, as well as a method for manufacturing the electrochemical sensor, which can overcome the shortcomings of devices and methods known in the art. In some embodiments, the electrochemical sensor is a microsensor. As used herein, the terms “sensor” and “microsensor” can be used interchangeably. As used herein, the terms “sensor” and “screen-printed electrode” can be used interchangeably. As used herein, the terms “screen-printed electrode” and “screen-printed, solid contact ion-sensitive electrode” can be used interchangeably. In embodiments, the term “screen-printed electrode” can refer to an array of electrodes comprising a working electrode, a reference electrode, and a counter electrode. For example, all devices described herein that can detect and/or measure electrochemical signals are sensors.

Aspects of the disclosure are drawn towards screen-printed, solid contact ion-sensitive electrode (SP-SC-ISE), comprising a screen-printed electrode, wherein the electrode is coated with at least two layers, wherein at least one layer comprises a conducting layer, and wherein at least one layer comprises an ion-sensing composite membrane.

In embodiments, the membrane comprises about 1 μm to about 1000 μm. In embodiments, the conducting layer comprises about 1 μm to about 20 μm. However, the thicknesses of the conducting layer and/or ion-sensing composite membrane can be determined by one of ordinary skill in the art to allow interaction between the sensing layer and the ion in the sample without being too thick to hinder the sensor's sensitivity.

In some embodiments, the SP-SC-ISE is disposable. In some embodiments, the SP-SC-ISE is paper-based.

In non-limiting, exemplary embodiments, the screen-printed electrodes can have all three electrodes—working, reference, and counter—integrated into a single compact unit. For example, the sensor can have all the electrodes pre-arranged for simple use without needing to assemble multiple components. In embodiments, the design of screen-printed electrodes can make them user-friendly. Everything is integrated into a single unit that can be disposable. The screen-printed electrode can be placed in contact with the sample (such as a drop of tear fluid) for measurement. Since the screen-printed electrode array is thin, flexible, and compact, it can easily be incorporated into portable diagnostic devices. This can be especially helpful in applications like testing for dry eye disease, where a quick and easy measurement is needed. In embodiments, a portable device can be connected to the SPE (Screen-Printed Electrode). In embodiments, the device can be portable or non-portable. The SPE can provide a quick, non-invasive way to test biological fluids (like tears) using the electrochemical signals generated by the sensor's electrodes.

In embodiments, the screen-printed electrode is selected from a carbon electrode or a glass carbon electrode.

In embodiments, the conducting layer comprises one or more conductive polymers, nanoparticles, or composite thereof. For example, the conductive polymer or composite thereof can be selected from poly (3,4-ethylene dioxythiophene) polystyrene sulfonate (PEDOT:PSS), graphene oxide (GO), PTh Polythiophene (PTh), and Polyacetylene (PA), or a combination thereof. However, other conductive polymers as known in the art can also be used. In embodiments, the nano-particles can comprise gold nanoparticles or other conductive nanoparticles as known in the art.

In embodiments, the conducting layer is deposited on the surface of the electrode. In embodiments, the ion-sensing composite membrane is deposited on the conducting layer.

In embodiments, the ion-sensing composite membrane comprises a polymer, solvent mediators, an ionophore, and a solvent. In embodiments, the polymer comprises polyvinyl chloride (PVC). In embodiments, the ionophore comprises an Na⁺ ionophore. In embodiments, the Na⁺ ionophore comprises lipophilic sodium tetrakis (4-fluorophenyl) borate dehydrate Na-ionophore. In embodiments, the solvent comprises tetrahydrofuran (THF). In embodiments, the solvent mediators can be selected from the group consisting of 2-nitrophenyl octyl ether (O-NPOE), Dioctylsebacate (DOS), di-2-ethylhexyl phthalate (DEHP), or any combination thereof. However other polymers, Na⁺ ionophores, solvent mediators, and solvents known in the art can also be used.

In embodiments, the ion-sensing, composite membrane has less than about 0.1% to about 10% cross-sensitivity to contaminants. For example, the ion-sensing composite membrane has about 0.01%, about 0.025%, about 0.05%, about 0.075%, about 0.1%, about 0.25%, about 0.5%, about 1.0%, about 2.5%, about 5%, about 7.5%, or about 10% cross-sensitivity to contaminants.

Aspects of the disclosure are drawn towards an electrochemical sensor for measuring the osmolarity of a sample comprising one or more electrodes and a sensing membrane, comprising a conductive layer and an ion-sensing membrane. As used herein, the terms “sensing-membrane” and “ion-selective conductive membrane” can be used interchangeably. As used herein, the term “sensing membrane” can refer to a membrane comprising a conductive layer as described herein, and an ion-sensing membrane as described herein.

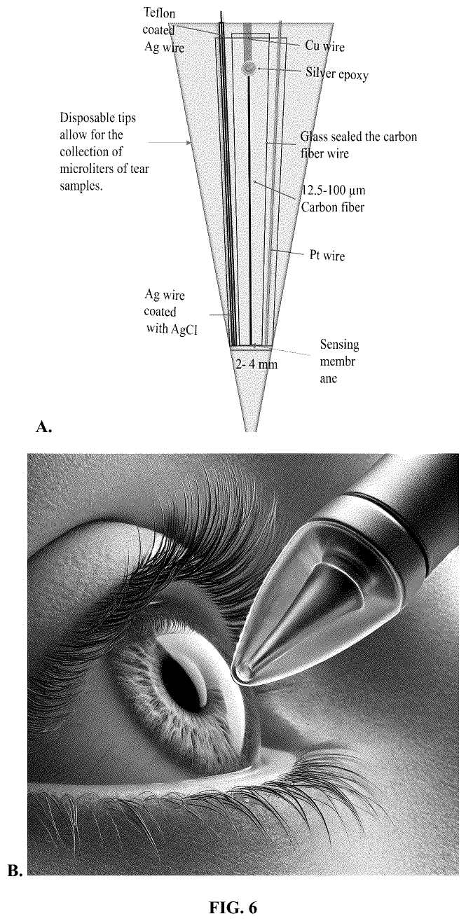

In some embodiments, the sensing membrane coats only the working electrode. For example, see FIG. 11. In some embodiments, the sensing membrane is in contact with the working electrode, the reference electrode, and the counter electrode. For example, see FIG. 6 panel a.

In embodiments, the sensor is a microsensor. In some embodiments, the sensor is disposable. In non-limiting, exemplary embodiments, see FIG. 11. In some embodiments, the sensor is reusable. In some embodiments, the sensor is reusable and can attach to disposable probes/tips for sample collection. In non-limiting, exemplary embodiments, see FIG. 6. For example, the reusable sensor can be detached and reattached to disposable probes/tips for each new sample. For example, the disposable probes/tips are single use items that interact directly with the sample and provide contamination-free collection of the sample.

In some embodiments, the one or more electrodes are screen-printed (see, e.g., FIG. 11). In some embodiments, the one or more electrodes can be fabricated by pulling carbon fiber (see, e.g., FIGS. 5 and 6). For example, we can insert a platinum wire as the working electrode (with a diameter ranging from about 10 μm to about 100 μm, for example, about 20 μm) and an Ag/AgCl reference electrode (can be about 10 μm to about 50 μm in diameter) into the pulled pipette to fabricate the microsensor. The counter electrode can be made of a material like platinum or carbon. In embodiments, the counter electrode can be a similar size to the working electrode.

In embodiments, the platinum wire can be used as the working electrode, where the electrochemical reactions related to the target analyte occur. The size range of 10 μm to 100 μm (with 20 μm as an example) is reasonable for this electrode, as it needs to be small enough to ensure localized measurements while still providing enough surface area for electrochemical reactions. For example we can insert a platinum wire (with a diameter ranging from about 10 μm to about 100 μm, for example, about 20 μm) and a reference electrode (the Ag/AgCl reference electrode can typically be as small as 50 μm in diameter, but it can go down to 10 μm for small systems like those in microtubes) into the pulled pipette to fabricate the microsensor.

For example, the sensor can comprise a carbon working electrode (WE), a silver reference electrode (RE), and a carbon counter electrode (CE).

The term “analyte” can refer to a compound being present in a fluid sample, wherein the presence and/or the concentration of the analyte can be of interest to the subject or to a medical staff, such as to a medical doctor. In embodiments, the analyte can comprise at least one ion. For example, the ion can comprise sodium (Na⁺).

An “electrochemical sensor” can refer to a sensor that can perform at least one electrochemical measurement, such as a plurality or series of electrochemical measurements, to detect the at least one substance as comprised within the body fluid by using an electrochemical method. In embodiments, the terms “electrochemical” and “electroanalytical” can be used interchangeably. For example, “electrochemical measurement” can refer to the detection of an electrochemically detectable property of a substance, such as an electrochemical detection reaction, by employing electroanalytic methods. In embodiments, electroanalytical methods can comprise potentiometry, coulometry, voltammetry, and amperometry. Thus, for example, the electrochemical detection reaction can be detected by applying and comparing one or more electrode potentials. The electrochemical sensor can be adapted to generate at least one electrical measurement signal which can directly or indirectly indicate a presence and/or an extent of the electrochemical detection reaction, such as at least one current signal and/or at least one voltage signal. The measurement can be a qualitative and/or a quantitative measurement.

In embodiments, the electrochemical sensor is an electrochemical sensor for the measurement of osmolarity. As used herein, the term “osmolarity” can refer to the number of milliosmoles of solute per liter of solution. As used herein, the terms “osmolarity” and “osmotic concentration” can be used interchangeably.

In embodiments, the sensor is disposable. As used herein, the term “disposable” can refer to an article that can be used once, or more than once until no longer useful, and then thrown away. In some embodiments, the sensor is reusable. For example, in some embodiments, the sensor can be reusable but have disposable components for patient sample collection. For example, the sample is tears from a patients eye.

The electrochemical sensor can be an amperometric sensor and/or a voltammetric sensor. An electrochemical sensor is one that functions by the production of a current when a potential is applied between at least two electrodes. The current generated can be proportional to the concentration of chemical species in solution. An amperometric sensor is a sensor that measures the current at a constant potential, and a voltammetric sensor is a sensor that measures current as a function of applied potential.

As used herein, the term “amperometry” can refer to measuring the electric current between a pair of electrodes that are driving an electrolysis reaction. For example, the current is proportional to the concentration of an analyte.

As used herein, the term “voltammetry” can refer to techniques in which the relation between current and voltage is observed during an electrochemical process. As used herein, the term “voltammogram” can refer to a graph of current vs. potential. In embodiments, voltammetry can comprise ion stripping voltammetry, cyclic voltammetry, and linear sweep voltammetry. In embodiments, the voltammetry can comprise square wave voltammetry. In embodiments, the voltammetry can comprise ion transfer voltammetry or ion exchange voltammetry. In embodiments, “cyclic voltammetry” can refer to a potentiodynamic electrochemical measurement wherein a working electrode potential is cycled, and the resulting current is measured. In embodiments, “linear voltammetry” can refer to a voltammetric method wherein the current at the working electrode is measured while the potential between the working electrode and reference electrode is swept linearly in time. In embodiments, “square wave voltammetry” can refer to a differential technique that uses a combined square wave and staircase potential applied to a working electrode. In embodiments, the electrochemical sensor can be an amperometric sensor or a voltammetric sensor.

In embodiments, the electrochemical sensor can use “ion-transfer stripping voltammetry (ITSV)”. As used herein, “ion-transfer stripping voltammetry (ITSV)” can refer to applying stripping voltammetry at the interface between two immiscible electrolyte solutions (ITIES). This can provide a highly sensitive technique for detection at sub-nanomolar limits. This method can be applied in environmental, industrial, food safety, and medical fields, including biomedical diagnostics. Molecular dynamics studies of the water/organic interface provide insights into its structure and the impact of molecular polarizability on ITIES interfacial properties. Ion-transfer stripping voltammetry (ITSV) is highlighted as a powerful technique, showcasing high sensitivity and selectivity for a broad range of organic and inorganic analytes. For example, without wishing to be bound by theory, ITSV can be applied in industrial and miniaturized sensor technology development for trace analysis. For example, ITSV can be used when the target ion is not easily reducible or oxidizable. The ion is absorbed into the ion-selective membrane, while an active and redox-conducting organic compound moves within the inner membrane. In the case of sodium ion detection, poly (3,4-ethylene dioxythiophene) polystyrene sulfonate (PEDOT) can act as the organic compound, reducing at the electrode to maintain charge neutrality.

Embodiments of the electrochemical sensor can comprise one or more electrodes. An “electrode” can refer to a part of the electrochemical sensor which can contact the fluid sample, directly or via at least one semipermeable membrane or layer.

In embodiments, the sensor can comprise a counter electrode, a working electrode, a reference electrode, or a combination thereof. In embodiments, the at least one electrode can be embodied in a manner that oxidative processes and/or reductive processes can take place at selected surfaces of the electrode.

A “counter electrode” can refer to an electrode that ensures that the correct potential difference between the reference electrode and the working electrode is being applied. For the example, the counter electrode can be any inert material, such as copper, carbon, ruthenium, rhodium, palladium, silver, rhenium, osmium, iridium, platinum, gold, graphite, and combinations and alloys thereof. In embodiments, the counter electrode is a platinum electrode or a carbon electrode.

A “working electrode” can refer to an electrode on which a reduction or oxidation reaction occurs. For example, the working electrode can be gold or carbon. For example, the working electrode can be paper or paper-based. For example, the working electrode can be a 3 mm gold working electrode.

In embodiments, the electrochemical sensor can further comprise a reference electrode. For example, electrical potential can be applied between the working electrode and the reference electrode, and raw current generated thereby can be measured between the working electrode and the counter electrode.

A “reference electrode” can refer to an electrode that has a stable and well-known electrode potential. For example, the reference electrode can be silver (Ag), silver/silver chloride (Ag/AgCl) electrode or saturated calomel electrode (SCE). In other non-limiting embodiments, the reference electrode can be a standard hydrogen electrode (SHE), a reversible hydrogen electrode (RHE), copper-copper (II) sulfate electrode, or a palladium-hydrogen electrode. For example, the reference electrode can be silver (Ag).

In embodiments, the working electrode can be coated in the sensing membrane. In embodiments, the working electrode, counter electrode, and reference electrode can all be in contact with the sensing membrane.

In embodiments, the electrodes described herein can comprise screen-printed electrodes (SPEs). As used herein, the term “screen printed electrode” can refer to an electrochemical device that is manufactured by printing inks on substrates. In embodiments the inks can comprise carbon, silver, gold, platinum, or a combination thereof. In embodiments, the substrates can comprise plastic, paper, metal, ceramic substrates, or a combination thereof. For example, the SPE comprises a paper-based SPE. For example, the paper-based SPE can be coated in gold. For example, the paper-based SPE can be coated in gold nanoparticles. In embodiments, the SPE can comprise a Conductive polymer.

In embodiments, the electrodes described herein can comprise a paper-based electrode. For example, the paper-based electrode can comprise paper-supported electrodes and paper-like electrodes. For example, the paper-based electrode can be constructed from materials and processes know in the art, see, e.g., Yao et al., Advanced Science, 2017, 4, 1700107. For example, the paper-based electrode can comprise a gold and/or carbon-based paper. For example, the paper-based electrode can comprise a cellulose-based paper. In embodiments, the paper-based electrode can be coated in a polymer, a metal, or combination thereof.

In embodiments, the sensors described herein can comprise a paper-based sensor. For example, the paper-based sensor can comprise a gold/carbon-based paper or other carbon-based materials. For example, the carbon-based materials can comprise carbon nanotubes, graphene, carbon-black, or other carbon-based materials known in the art.

In embodiments, the SPE can be non-paper-based. For example, the SPE can be printed on other solid materials as known in the art.

In embodiments, the electrode is a screen-printed, solid contact ion-sensitive electrode (SP-SC-ISE), comprising a screen-printed electrode, wherein the electrode is coated with at least two layers, wherein at least one layer comprises a conducting layer, and wherein at least one layer comprises an ion-sensing membrane composite.

In some embodiments, one or more of the electrodes are not screen-printed. For example, the electrodes can be fabricated from carbon fibers and metal wires (see, e.g., FIG. 6 panel a). For example, the working electrode can be fabricated from pulling a carbon fiber wire in a glass and/or quartz capillary (see, e.g., FIG. 5). In embodiments, after pulling the carbon fiber to the desired size, an inert metal wire can be inserted as the counter electrode. For example, the wire can be a platinum (Pt) wire, which is commonly used due to its stability and conductivity in electrochemical measurements. In some embodiments, a reference electrode can also be inserted into the pulled carbon fiber. For example, the reference electrode can be an Ag wire coated with Ag/AgCl (silver chloride), and then further coated with a material to provide insulation and prevent interference from unwanted ions. For example, the material can be polytetrafluoroethylene (PTFE) or other similar materials as known in the art.

In embodiments, the microsensor is placed in a tip designed to suction the solution toward the microelectrode. For example, the tip can be a probe for diagnostic or monitoring purposes. For example, the sensor can be a microfluidic suction probe and/or flow-through sensor. For example, the microsensor can be a small, precise sensor designed to interact with the sample solution in a diagnostic or monitoring context. The integration of the sensor into a tip is useful because it allows the sensor to be easily positioned in close contact with the solution (for example, tear fluid) to detect specific analytes such as Nat. Suction Functionality: The tip is designed to suction the solution toward the microelectrode to ensure that the sample is drawn into direct contact with the sensor. This enhances the accuracy of measurements by providing a continuous, consistent sample flow into the sensing region. The suction feature also minimizes sample waste and helps in environments where it's important to capture small, localized fluid samples (like microfluidic applications or tear fluid testing). Probe for Diagnostic or Monitoring: The tip, combined with the microsensor, can be used as a probe for in vivo or in vitro testing. For diagnostic purposes, this means it could be used for point-of-care applications where a small amount of fluid is analyzed, providing real-time information. The sensor's ability to suction and collect the sample ensures that only the relevant analyte is tested, making it especially useful in personalized or targeted diagnostics.

In embodiments, the sensor can further comprise silver epoxy and copper wire, e.g., FIG. 6 panel a. These components can be used to stablize electrical connections, mechanical support, and precise signal measurement. The silver epoxy can create electrical connections in electrochemical devices. It's a conductive adhesive that can prove a reliable bond between components like the electrodes and other parts of the microsensor. In the context of the sensor, silver epoxy can be used to secure the microelectrode to the sensor body or to connect the electrode to the necessary electrical leads. This can be useful when for small, sensitive components like microelectrodes, where a traditional soldering process is not feasible. The copper wire can serves as a connector to the electrodes, transmitting the electrical signals generated by the electrochemical reactions occurring at the working electrode. Copper is a highly conductive material, which allows the sensor to efficiently transfer the signals to the external measurement system.

In FIG. 6 panel a, the copper wire can act as the electrical lead that connects the microelectrode (working, counter, or reference) to an external circuit for signal processing and measurement. The copper wire can serve as a connector to the electrodes, transmitting the electrical signals generated by the electrochemical reactions occurring at the working electrode. Copper is a highly conductive material, which allows the sensor to efficiently transfer the signals to the external measurement system. For example, in FIG. 6 panel a, the copper wire can act as an electrical lead that connects the microelectrode (working, counter, or reference) to an external circuit for signal processing and measurement.

As used herein, the term “microfluidic suction probe” can refer to can refer to a device designed to extract or draw in small volumes of liquid or fluid samples through suction, typically using a microelectrode or sensor integrated into the probe. The probe is capable of creating a localized flow of fluid toward the sensor for the purpose of chemical, biological, or electrochemical analysis. The term “microfluidic” can refer to a the probe operates on a microscale, where precise control over fluid movement and small volumes is required, often in diagnostic or monitoring applications.

In embodiments, the conducting layer comprises a conductive polymer or composite thereof. In embodiments, the conductive polymer of composite thereof poly (3,4-ethylene dioxythiophenc) polystyrene sulfonate (PEDOT:PSS), graphene oxide (GO), PTh Polythiophene (PTh), and Polyacetylene (PA), or a combination thereof.

In embodiments, the ion-sensing membrane composite can comprise a polymer, solvent mediators, an ionophore, and a solvent. For example, the polymer can comprise polyvinyl chloride (PVC) and Poly (vinyl acetate). For example, the polyvinyl chloride (PVC) and Poly (vinyl acetate) can comprise about Mw ˜100,000. However, other molecular weights as known in the art can be used.

In embodiments, the ionophore comprises a Na⁺ ionophore. For example, the Na⁺ ionophore comprises lipophilic sodium tetrakis (4-fluorophenyl) borate dehydrate Na-ionophore.

In embodiments, the solvent can comprise tetrahydrofuran (THF). However, other solvents known to those of ordinary skill in the art can be used. For example, the solvent can comprise a polar, aprotic solvent. For example, the solvent can comprise a polar, protic solvent. For example, the solvent can comprise a nonpolar solvent.

In embodiments, the conducting layer and ion-sensing layer can be drop cast on the surface of the electrode. As used herein, the term “drop cast” can refer to the formation of a film by dropping a solution onto a surface followed by the evaporation of the solution.

In embodiments, the conducting layer is drop cast on the surface of the electrode. In some embodiments, the conducting layer is drop cast on the top of the ion-sensing layer. In embodiments, the ion-sensing membrane is drop cast on the surface of the electrode. In some embodiments, the ion-sensing membrane is drop case on top of the conducting layer.

As used herein, the terms “layer”, “membrane”, and “coating” can be used interchangeably.

In embodiments, the one or more electrodes can be surrounded by or partially surrounded by a biocompatible coating. The coating can provide a biocompatible interface between the sensor and the fluid sample that wicks the sample over the electrodes. In embodiments, the entirety of the electrode can be surrounded by the coating. In embodiments, about 10%, about 20%, about 30%, about 40%, about 50%, about 60%, about 70%, about 80%, about 90%, or about 100% of the electrode can be surrounded by the coating. In embodiments, only about the portion of the electrode that is in contact with the fluid sample can be surrounded by the coating.

In embodiments, the coating can be deposited on the surface of one or more electrodes of the electrochemical sensor. For example, the coating can be deposited onto the electrodes by spin coating, immersion, electrodeposition, or electropolymerization.

In an embodiment, for example, a working electrode can be polished with an alumina paste slurry on a polishing pad prior to the coating being deposited onto the electrode by spin coating. After spin coating, the coating was dried at room temperature for a period of time, such as 1 hour. In embodiments, a conductive polymer, can be deposited onto the coating by spin coating.

In another embodiment, for example, a working electrode can be immersed in a coating comprising a polymer, such as aniline, and then the coating can be immobilized on the electrode, such as by electropolymerization. Variability of membrane thickness can be achieved by adjusting the applied potential during electropolymerization.

In another embodiment, for example, the Screen-Printed Electrodes (SPEs) can be subjected to an electrodeposition procedure. In embodiments, a composite material can be deposited on the working electrode. In embodiments, the composite material comprises a conductive polymer or composite thereof.

“Conductive polymer” can refer to a polymer which can conduct electricity. The electrical conductivity can be tuned depending on the type of polymer(s) used. Non-limiting examples of such conductive polymers comprise a perfluorosulphonic acid polymer, 3,4-ethylene dioxythiophene (EDOT), Polythiophene (PTh), Polyacetylene (PA), polyaniline (PANI), pyrrole, polypyrrole, and/or Poly (3,4-ethylenedioxythiophene)-poly (styrenesulfonate) (PEDOT:PSS). For example, the conductive polymer can be Sulfonated Tetrafluoroethylene (Nafion™). For example, the conductive polymer can comprise polypyrrole and polyaniline. For example, the conductive polymer can comprise poly(3,4-ethylenedioxythiophene)-poly(styrenesulfonate) (PEDOT:PSS). conductive polymer or composite thereof comprises poly (3,4-ethylene dioxythiophene) polystyrene sulfonate (PEDOT:PSS), Polythiophene (PTh), and Polyacetylene (PA), or a combination thereof.

In embodiments, the conductive polymer can be deposited on the coating that surrounds the one or more electrodes. For example, the conductive polymer can be deposited onto the coating that surrounds the one or more electrodes by spin coating, immersion, or electropolymerization. As used herein, the coating surrounding one or more electrodes can be referred to as a “membrane”. As used herein, the terms “coating” and “membrane” can be used interchangeably.

In other embodiments, the conductive polymer can be admixed within the coating prior to the coating being deposited on the one or more electrodes.

Electrode fouling can involve the passivation of an electrode surface by an anti-fouling agent that forms an increasingly impermeable layer on the electrode, thereby inhibiting direct contact of an analyte of interest with the electrode surface for electron transfer. Embodiments as described herein can comprise compositions and methods to prevent electrode fouling, for example incorporating an anti-fouling polymer into the coating. Non-limiting examples of anti-fouling polymers comprise poly (ethylene glycol) (PEG), zwitterionic polymers, poly (hydroxyfunctional acrylates), poly (2-oxazoline) s, poly (vinylpyrrolidone), poly (glycerol), peptides and peptoids.

In embodiments, the coating has a thickness of about 100 μm to about 1 mm. For example, the thickness of the coating can be less than about 100 μm thick. For example, the thickness of the coating can be about 100 μm, about 200 μm, about 300 μm, about 400 μm, about 500 μm, about 600 μm, about 700 μm, about 800 μm, about 900 μm, or about 1 mm. For example, the coating can be greater than about 1 mm. For example, the thickness of the coating can be controlled by the electropolymerization cycle. That is, variability of membrane thickness can be achieved by adjusting the applied potential during electropolymerization.

In embodiments, the one or more electrodes can be present in a microfluidic device in communication with a microfluid channel. A “microfluidic device” can refer to a device comprising fluidic structures and internal channels having microfluidic dimensions. These fluidic structures can include chambers, valves, vents, vias, pumps, inlets, nipples, and a detection means, for example. Microfluidic channels can comprise fluid passages having variable length and at least one internal cross-sectional dimension that is less than approximately 500 μm to 1000 μm, such as between approximately 0.1 μm and approximately 500 μm.

For example, the sensors described herein can be in communication with an electrochemical microfluidic device as known in the art. For example, FIG. 11 shows a sensor described herein in communication with an electrochemical microfluidic device.

In embodiments, electrochemical sensor can comprise the one or more electrodes immobilized on a solid support member. Non-limiting examples of the composition of the solid support member comprise plastic, cardboard, glass, plexiglass, tin, paper, or a combination thereof.

In embodiments, the support member comprises a screen-printed member. For example, embodiments comprise a screen-printed electrode (SPE) based sensor, which has advantages such as robustness, excellent detection limitations, selectivity, sensitivity portable, cost-effective with potential of mass production and this electrode in paper based.

In embodiments, the sensor can be a microelectrode. In embodiments, the inner diameter of the microsensor is less than about 5 mm, about 5 mm, about 10 mm, about 15 mm, about 20 mm, about 25 mm, about 30 mm, about 35 mm, about 40 mm, about 45 mm, about 50 mm, about 60 mm, about 75 mm, about 100 mm, or greater than about 100 mm.

In embodiments, the electrochemical sensor can be miniaturized, thereby improving the portability, case of handling, case of incorporation into arrays, and the like. The term “miniaturization” can refer to a manufacturing technique to reduce the size of the electrochemical sensor.

In embodiments, the integration of compositions, processes, and methods described herein can provide increased sensitivity and selectivity, increased stability, cost-effectiveness and accessibility, miniaturization, longevity and reduced drift, improved signal detection, and closed-loop integration. For example, increased sensitivity and selectivity can refer to the ability to detect sodium accurately and selectively. For example, stability can refer to consistent performance over time and various conditions. For example, cost-effectiveness and accessibility can refer to creating an affordable product that is accessible to a wider population. For example, miniaturization can increase convenience for wearability. For example, longevity and reduced drift can extend the senor's lifespan and minimize measurement errors. For example, improved signal detection can enhance the detection and interpretation of osmolarity.

Aspects of the invention are further drawn to a “sensor system.” A sensor system can refer to a device which is configured for conducting at least one medical analysis. For example, the sensor system can be a device configured for performing at least one diagnostic purpose and/or a monitoring purpose, such as a system comprising at least one electrode as described herein for performing the at least one medical analysis. The sensor system can, for example, comprise an assembly of two or more components that can interact with each other, for example to perform one or more diagnostic and/or monitoring purposes, such as to perform the medical analysis. Specifically, the two or more components can perform at least one detection of the at least one analyte in the body fluid and/or in order to contribute to the at least one detection of the at least one analyte in the body fluid.

### Method of Detection

Aspects of the invention are drawn towards methods of detecting osmolarity. Further aspects of the invention are drawn towards methods of diagnosing dry eye disease.

Further aspects of the invention are drawn towards methods of measuring or determining sodium (Na⁺) using voltammetry. For example, ion transfer stripping voltammetry.

A “fluid sample” can refer to any liquid sample containing or suspected of containing the analyte(s) of interest. In embodiments, the fluid sample can be a body fluid. In other embodiments, the fluid sample can be a consumable.

In embodiments, the fluid sample can be a body fluid. The term “body fluid” can refer to a fluid present in a body or a body tissue of a subject and/or which can be produced by the body of the subject. For example, the body fluid can be tear fluid. However, additionally, or alternatively, one or more other types of body fluids can be used, such as saliva, urine, ascites, cerebrospinal fluid (CSF), sputum, bone marrow, synovial fluid, aqueous humor, amniotic fluid, cerumen, breast milk, bronchioalveolar lavage fluid, semen, prostatic fluid, cowper's fluid or pre-ejaculatory fluid, female ejaculate, sweat, fecal matter, hair, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood, or other body fluids.

During the detection of the at least one analyte, the body fluid can be present within the body or body tissue. Thus, the sensor system can at least be configured for detecting the at least one analyte within the body tissue.

The methods described herein can involve obtaining a fluid sample from a subject. As used herein, the phrase “obtaining a fluid sample” can refer to any process for directly or indirectly acquiring a fluid sample from a subject. For example, a fluid sample can be obtained (e.g., at a point-of-care facility, e.g., a physician's office, a hospital, laboratory facility, at home by a patient) by procuring a tissue or fluid sample (e.g., tear fluid) from a subject. Alternatively, a fluid sample can be obtained by receiving the fluid sample (e.g., at a laboratory facility) from one or more persons who procured the sample directly from the subject.

In embodiments, the method can be repeated periodically. “Periodically” can refer to performing the method at recurring or repeating intervals, such as about every one minute, about every 5 minutes, about every 30 minutes, about every one hour, about every 4 hours, about every 8 hours, about every 12 hours, about every 24 hours, about once a day, about once a week, about once a month, about every three months, about every 6 months, or about every year.

In embodiments, the subject's tear osmolarity can be measured once, or can be measured continuously or periodically over a period of time. The term “period of time” can refer to the period of time necessary to achieve an effect or result. For example, the period of time can comprise about 1 hour, about 2 hours, about 3 hours, about 4 hours, about 5 hours, about 6 hours, about 12 hours, about 18 hours, about 24 hours, about 36 hours, about 48 hours, or longer than about 48 hours. In embodiments, the period of time comprises about 12 or about 24 hours.

The term “continuously” can refer to without interruption or with minimal interruption. For example, continuous measurement can refer to the fact that the measurements are repeated continuously over very small intervals of time.

In embodiments, the sensor has a lower detection limit of about 5 μM. As used herein, the term “lower detection limit” can refer to the lowest concentration or amount of a substance that can be detected. In embodiments the sensor has a lower detection limit of about less than about 1.0 μM, about 1.0 μM, about 5.0 μM, about 10.0 μM, about 15.0 μM, about 20.0 μM, or greater than about 20.0 μM. For example, the sensor's analyte concentration detection range is contemplated to be 0.026 +0.031 mM.

### Method of Diagnosing

Aspects of the invention are further drawn towards methods of diagnosing a subject with or at risk of a dry eye disease. In embodiments, the method comprises applying a sensor described herein to the tears of a subject, measuring the osmolarity of the tears, and determining whether the subject is afflicted with Dry Eye Disease (DED) based upon the osmolarity. In embodiments, the osmolarity can be determined by measuring the physical or electrochemical signal of a target analyte in the sample, converting the sensor signal to osmolarity by measuring the sensor signal (e.g., voltage, current, or resistance) at known concentrations of a reference solution to generate a concentration curve, and multiplying the concentration of the sodium ion by the appropriate van't Hoff factor n, accounting for dissociation. However, other methods of measuring the osmolarity can be used as known in the art.

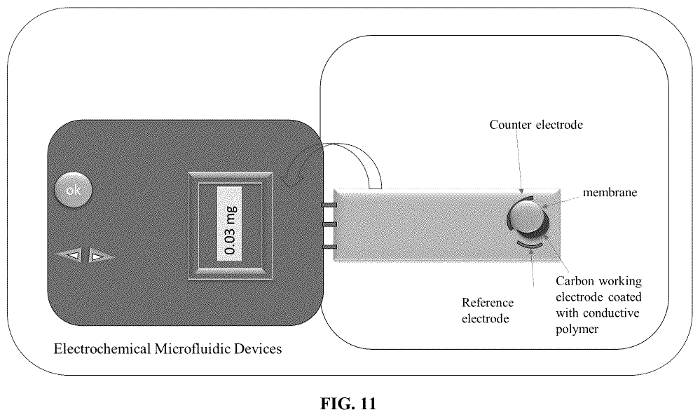

In embodiments, the osmolarity indicating dry eye will be levels known in the art by one of ordinary skill in the art (e.g., a clinician, an optometrist, an ophthalmologist, etc.). For example, the degree of dry eye can be indicated by the osmolarity. In non-limiting, exemplary embodiments, FIG. 10 shows osmolarity levels indicating no dry eye (“normal”), mild dry eye, moderate dry eye, and severe dry eye. For example, a normal eye can be indicated by an osmolarity of about 300 mOsm/L or less. Mild dry can be indicated by an osmolarity of about 300 mOsm/L to about 320 mOsm/L. Moderate dry eye can be indicated by an osmolarity of about 320 mOsm/L to about 340 mOsm/L. Severe dry eye can be indicated by an osmolarity of about 340 mOsm/L or greater.

In embodiments, the osmolarity can be determined by any electrochemical technique described herein. For example, the osmolarity can be determined by ITSV.

The term “diagnosing” can refer to determining presence or absence of a disease, classifying a disease or a symptom, determining a severity of the disease, monitoring disease progression, forecasting an outcome of a disease and/or prospects of recovery.

The term “subject” can refer to any organism to which aspects of the invention can be performed, e.g., for experimental, diagnostic, prophylactic, and/or therapeutic purposes. Example of subjects can include be mammals, such as primates, for example humans. For veterinary applications, a wide variety of subjects will be suitable, e.g., livestock such as cattle, sheep, goats, cows, swine, and the like; poultry such as chickens, ducks, geese, turkeys, and the like; and domesticated animals such as pets, for example dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject.

For example, embodiments can comprise exposing a fluid sample obtained from a subject to an electrochemical sensor as described herein, and detecting the current generated from the applied voltage during said exposing, wherein current corresponds to the osmolarity in the fluid sample.

Aspects of the invention are also drawn towards methods of monitoring a subject with or at risk of dry eye disease. The term “monitoring” can refer to the act of measuring, quantifying, qualifying, estimating, sensing, calculating, interpolating, extrapolating, inferring, deducing, or any combination of these actions. For example, “monitoring” can refer to a way of getting information via one or more sensing elements, such as an electrochemical sensor as described herein.

### Method of Preventing or Treating

Aspects of the invention are further drawn towards a method of preventing or treating a subject afflicted with or at risk of dry eye disease.

The term “prevent,” “prevention,” or “preventing” can refer to any method to partially or completely prevent or delay the onset of one or more symptoms or features of a disease, disorder, and/or condition, such as diabetes. Prevention can be administered to a subject who does not exhibit signs of a disease, disorder, and/or condition.

The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as dry eye disease. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

In embodiments, treatment can comprise administering to the subject one or more therapeutic agents. The term “therapeutic agent” can refer to any chemical moiety that is a biologically, physiologically, or pharmacologically active substance that acts locally or systemically in a subject. The term also can refer to any substance intended for use in the diagnosis, cure, mitigation, treatment or prevention of disease or in the enhancement of desirable physical or mental development and/or conditions in an animal or human. For example, embodiments can comprise exposing a fluid sample obtained from a subject to an electrochemical sensor as described herein, detecting the current generated from the applied voltage during said exposing, wherein current corresponds to the osmolarity in the fluid sample, and treating the subject for dry eye. In embodiments, the osmolarity indicating dry eye can be known by one of ordinary skill in the art.

### Kit

Aspects of the invention are also drawn towards a kit comprising the electrochemical sensor as described herein. The term “kit” can refer to a product (i.e., a kit of parts) comprising one package or one or more separate packages and including informational material. In embodiments, the kit can further comprise components and/or reagents that can measure osmolarity in a subject.

In embodiments, the kit can comprise one or more disposable articles for measuring tear osmolarity. The term “disposable article” can refer to a single or limited use article that is made from relatively inexpensive materials that make the article cost effective to fabricate. For example, the disposable article can be a swab, spoon, dipstick, filter paper, or test-strip.

In embodiments, the kit can comprise a medical device. The term “medical device” can refer any instrument, apparatus, implant, in vitro reagent or similar or corresponds article that is used to diagnose, prevent, or treat a disease or other condition, and does not achieve its purpose through pharmacological action within or on the body. For example, the medical device can be a sensor, such as an electrochemical sensor as described herein.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 11

### Example 1: Disposable Sensors and the Development of a New Ion-Selective Microelectrode Using Electrochemical Techniques for the Direct Detection of Tear Osmolarity

Introduction: In embodiments, the technology described herein addresses a critical need within the field of ophthalmology. With the increasing prevalence of Dry Eye Disease (DED) due to extended screen time and electronic device usage, the points and objectives of this research project can include, but are not limited to:

DED Diagnosis Challenge: DED is a common eye condition characterized by reduced tear production, elevated tear film osmolarity, and inflammation. Traditional diagnosis relying solely on symptoms is often unreliable since these symptoms can mimic those of other eye diseases.

Demand for Reliable Diagnosis: There is an urgent need for swift, cost-effective, and accurate methods to identify individuals at risk of DED. Timely diagnosis is vital for initiating prompt treatment and effectively managing the disease.

Non-Limiting, Exemplary Solution: Embodiments of the technology described herein are an innovative microsensor, accompanied by a screen-printed electrode (SPE), to detect tear osmolarity by measuring sodium ions (Na⁺) using voltammetry. For example, ion transfer stripping voltammetry will be employed in this microsensor.

Advantages of Voltammetry: Voltammetry, an electrochemical technique that records current in relation to applied voltage, can be harnessed to quantify the concentration of sodium ions in tears. An invaluable feature of this technique is its capacity to provide near-real-time measurements, enabling continuous monitoring.

We can manufacture a portable, disposable microsensor and SPE that can be mass-produced. These tools can offer increased detection capabilities, selectivity, and sensitivity for diagnosing DED. They can also be rapid-performing instruments, allowing swift and precise identification of individuals at risk of DED.

Impact: The development and implementation of these microsensors and SPEs can revolutionize DED diagnosis and management. They will provide eye care professionals with a valuable tool for early detection, thereby enhancing the quality of care for individuals affected by DED.

We can address the escalating concern of DED through an innovative microsensor and screen-printed electrode (SPE) capable of identifying tear osmolarity through sodium ion measurements as described herein. This breakthrough can enhance the accuracy of DED diagnosis and subsequently improve treatment outcomes.

Diagnosing Dry Eye Disease (DED) poses challenges. The widely used Schirmer test 30 has numerous disadvantages, including poor reproducibility, low sensitivity and specificity, and severe patient discomfort. The Tear Lab test, though available, is less common due to its high device cost, approximately $10,000 per test card at $14.00 per eye, and limited availability in only a few clinics and hospitals. Abnormal body fluids are crucial diagnostic information sources in clinical applications. Solid Contact Ion-Sensitive electrodes (SC-ISE) are virtual devices that monitor a patient's health status using body fluids and blood tests. Screen-Printed Electrodes (SPEs) offer a cost-effective, user-friendly approach, enabling rapid and efficient data collection. Moreover, they enable long-term and real-time monitoring, making them efficient devices for one-step, contamination-free measurements.

Diagnosing DED using physiological biomarkers, such as gene technology (to assess conditions like rheumatoid arthritis or thyroid problems that can cause DED), is challenging due to complex diagnostic processes and poor correlation between clinical signs and symptoms. Therefore, the direct, rapid, and accurate measurement of tear osmolarity using electrochemical sensors is crucial for DED diagnosis. The technology described herein aligns with the National Eye Institute (NEI)'s NOSI for diagnosing dry eye disease (NOT-EY-21-007) for the Anterior Segment Initiative, which aims to identify and develop new biomarkers and effective diagnostic methods for DED.

Described herein is the use of Ion-Transfer Stripping Voltammetry (ITIES) for DED diagnosis. This technique is new compared to conventional methods like voltammetry and potentiometry because:
1. It takes precedence over voltammetry, as it effectively deals with ions that are not readily oxidized or reduced.
2. ITIES improves the low sensitivity and interference issues associated with potentiometry.

Amperometric or voltammetric sensors enhance selectivity by adjusting the voltage of a measuring electrode to match the oxidation or reduction potential of the target analyte ion. However, ITSV, based on ion transfer, is crucial when the target ion isn't easily reducible or oxidizable. The desired ion is absorbed into the ion-selective membrane, while an active and redox-conducting organic compound moves within the inner membrane. In the case of sodium ion detection, poly (3,4-ethylene dioxythiophenc) polystyrene sulfonate (PEDOT) acts as the organic compound, reducing at the electrode to maintain charge neutrality. Successful investigations of ITSV for various ions, including antidepressants, lead, and chromium (VI), have been conducted. We employ a highly selective sodium ionophore (IV) to create a sodium ion-selective membrane. Ionophore (IV) is a 16-crown-5 derivative ionophore with a cavity suitable for Na⁺ ions, preventing complex formation with larger ions like potassium.

In some embodiments, the sensor described herein, using hydrophobic conductors with a large surface area and high double-layer capacity (employing nanostructured materials), can provide excellent stability for improving SC-ISE. While ISE designs have evolved from bulk electrodes with aqueous internal filling solutions to planar solid contact electrodes, challenges like sensor lifetime remain due to weak membrane stability. Herein, we utilize solid contacts for electrode miniaturization. Employing SC as operating parameters for ion-selective polymer membranes based on ionophores helps maintain membrane surface balance, reducing potential drifts. SPEs, modified in various ways, offer creativity in sensor fabrication. SPEs are considered among the best electrodes for in situ clinical analysis due to their fast response, design flexibility, small sample size, high sensitivity, and lack of pretreatment requirements. Despite the strong correlation between tear osmolarity and eye abnormalities, several methods for detecting it rely on expensive osmometers.

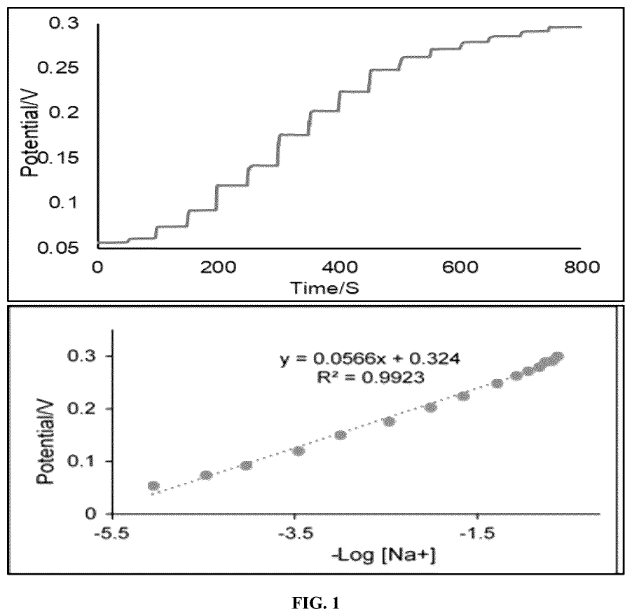

In some experiments, the open circuit potentiometry (OCP) technique was used for potentiometry measurements. A non-limiting, execmplary response of the SC-SP-ISE with thin membranes is illustrated in FIG. 1 panel (A), where the measurement procedure involved a solution ranging from 2.5×10⁻⁶ to 4.8×10⁻¹M NaCl in a phosphate buffer solution at pH 7.0 (PBS). FIG. 1 panel (A) displays the OCP characteristics of one of the electrodes and shows that the developed Na⁺-SC-SP-ISE exhibits a wide linear sensing range of 3.37×10⁻⁵ to 2.40×10⁻¹ mol/L or 6.74×10⁻⁵ to 4.80×10⁻¹ Osm/L. We employed thirteen individual sensors with identical compositions, yielding results with a near Nernstian slope of 57.2±1.35 (n=13) and a lower limit of detection of 0.026±0.031 mM, along with a linear correlation R²=0.9961±0.002 (n=13). FIG. 1 panel (B) displays a correlation coefficient for one of the measurements. Our studies indicated that the modified SPE using the OCP method can detect Na which can be used for monitoring tear osmolarity.

Construction of the SC-SP-ISE: We applied a carbon screen-printed electrode coated with two layers: a PEDOT:PSS layer drop-cast directly onto the carbon surface and allowed to dry at room temperature. Subsequently, the ion-sensing membrane (ISM) composite was drop-cast evenly onto the same surface of the electrode. The ISM composite was prepared using a mixture of polyvinyl chloride (PVC), solvent mediators, lipophilic sodium tetrakis (4-fluorophenyl) borate dehydrate Na-ionophore, and tetrahydrofuran (THF). The mixture was sonicated for 30 minutes before use. After each coating, the membrane was air-dried for 12 hours. SPEs were then conditioned for 60 minutes in a 10⁻³ M solution of NaCl.

### Sensor for Monitoring Dry Eyes

In addition to using screen-printed electrodes, we can use the microsensor described herein for monitoring dry eyes. The Electrochemical Medical Sensors market size was $26.8 million in 2019, accounting for 71% of the market in 2021. The electrochemical sensors market is expected to grow by 6.65% in 2030. Meanwhile, the size of the medical sensors market was $13.7 billion in 2022 and is expected to reach $25.8 billion by 2030, growing at a rate of 7.33%. The Ion-Selective Electrode (ISE) is commonly used in the potentiometric technique, where the open circuit potential is recorded against a reference electrode under zero current conditions. The electrochemical sensor described herein can measure Na⁺ based on ion transfer stripping voltammetry, a new use of PEDOT-PSS embedded membrane and an ionophore selective to sodium. With this combination, without wishing to be bound by theory, we can measure an unprecedented range of sodium concentrations in tears.

The osmolarity monitoring microsensor described herein can comprise at least three innovations:

Ion-transfer stripping voltammetry to increase the measurement range, especially on the low end, compared to a potentiometric sensor or voltammetric sensor operating in a traditional mode.

A new PEDOT:PSS coated electrode that increases the loading of the analyte and improves the signal-to-noise ratio and limit of detection.

Translating an analyzer based on ITSV into a probe that can be used outside the medical clinic.

The sum of these three technical innovations can result in a real-time osmolarity monitoring microsensor at a price point lower than competing methods.

We have constructed the working prototype. The electrochemical analyzer can comprise of three electrodes: a working electrode, a counter electrode, and a reference electrode. The developments can comprise the probe coated with PEDOT:PSS, the thickness of the conductive layer, and the performance of the PVC coating and ionophores.

Additionally, three separate electrodes can be examined through a single probe. The signal generator and data recorder can be included in one analyzer. The voltammetry circuit can be translated and measure the resulting voltammogram.

Non-limiting measurements on sodium indicate that the method can detect sodium. Wc applied sodium (IV) ionophore 24, highly selective for sodium, to create a membrane selective for sodium ions. Ionophore (IV) is a 16-crown 5-derivative ionophore with a Na⁺-sized cavity that prevents complexation with larger ions such as potassium. Evaluating the relationship between ionophore chemical structures and ion selectivity properties helps host-guest chemistry develop a highly selective ionophore for analytes.

FIG. 3 shows Ion-Transfer Stripping Voltammetry (ITSV) on the modified 3 mm glass electrode (GCE). Upon the application of adequate negative potential to the GCE, its PEDOT:PSS conductive polymer coating becomes reduced while the positively charged sodium ions are transported into the PVC composite membrane. During this process, the plasticized PVC membrane serves as a reservoir for the Na⁺ ions, which become preconcentrated in the PVC membrane from the aqueous solutions. In this process, the PEDOT: PSS conductive polymer film acts as an ion-to-electron transducer to mediate continuous current flow between the conductive membrane and the electronically conductive solid electrode. Applying 0.05 V for 7 minutes of preconcentration, the sodium ions are stripped from the PVC membrane by linearly sweeping the potential of the GCE toward positive potential values. At positive potentials, the PEDOT:PSS film becomes oxidized, driving the transfer of Na⁺ ions from the membrane into the aqueous phase.

As shown in FIG. 3 panels (a, b), the stripping current was proportional to the concentration of sodium in the range of 0.6 mM to 257 mM of NaCl solution, which corresponds to an osmolarity of 2 mOsmol/L to 576 mOsm/L. The R² values of the regression lines that fitted to the calibration curve for sodium was 0.9943. A least-square analysis of pcak currents versus Na⁺ concentrations in sample solutions was applied to determine the slope, m, intercept, b, and standard deviation about regression, Sr, as given by the following equations (N is the number of standard solutions). N−2 represents the degrees of freedom. The lower limit of detection (LOD) for sodium ion was calculated as 28 μM (28×10∧−6 M) using the following equation: Lower detection limit≡3 Sr/m.

The development of a microsensor for diagnosing dry eye disease (DED) presents significant commercial opportunities. DED affects millions of people worldwide, and the market for diagnostic and treatment technologies for DED is growing rapidly.

The microsensor technology described herein offers several advantages over traditional diagnostic methods for DED, such as the Schirmer test, which involves placing a paper strip in the eye to measure tear production. The microsensor technology provides a more accurate and objective measure of tear osmolarity, enabling early identification of DED and more targeted treatment plans. According to current research, the global dry eye syndrome market is estimated to reach $600 million by the end of 2035, growing at a CAGR of 8% during the forecast period from 2023 to 2035. This growth can be attributed to the increasing number of people suffering from dry eye syndrome and the higher costs of its treatment. Additionally, dry eye is often associated with various other diseases such as thyroid disorders, diabetes, and arthritis, further boosting market growth. Therefore, there is a high potential market for our microsensor technology, including optometrists, ophthalmologists, and medical device companies. The market for DED diagnostic and treatment technologies is expected to reach $6.6 billion by 2027, driven by the increasing prevalence of DED and the demand for more effective and personalized treatment options.

Moreover, the microsensor technology can reduce healthcare costs by enabling early identification and treatment of DED presents an attractive value proposition for healthcare providers, insurers, and patients. By improving the accuracy of DED diagnosis and facilitating more targeted treatment plans, microsensor technology has the potential to reduce healthcare costs.

Based on studies conducted on the impact of DED on the quality of life in Canadian patients, DED has been assessed as moderate or severe by 19.2% and 69.2% of patients, respectively. The mean total DED per annual patient costs were $24. This includes three office visits per year per patient costing $240, Tear Lab tests at $22 per year with a device cost of $10,800, external photos at $20 per year with a device cost of $6,000, Inflammatory tests at $11.90 per year with a device cost of $6,000, Punctal occlusions costing $140 for the first eye and $70 for the second eye with a device cost of $3,259, and Intense Pulsed Light treatments costing $800 to $1,000 for three to four sessions per year. Other methods, such as meibography, also incur costs for patients.

The pain caused by DED can be compared to a type of chronic pain syndrome that has a detrimental effect on the mental and physical health of patients and their quality of life. Advances in the understanding of dry eye can lead to better and faster treatments for the millions of people affected by the condition in the United States and worldwide. The high-speed/low-cost microsensor described herein for diagnosing this disease can avoid the need for lengthy and complicated treatments and eye infections. Ophthalmologists refrain from routinely performing the diagnosis of this disease due to limitations. Failure to promptly measure dry eye can negatively affect visual quality and lead to darkening of the stroma and cause pain. The current diagnosis of DED is based on using the Schirmer test, which involves many disadvantages, including poor reproducibility, low sensitivity and specificity, and severe patient discomfort. A Tear Lab test is also available, which is less common due to its high cost of about $10,000 for the device, and each test card costs $14.00.

### Example 2

### Example 2: New Ion-Selective Microelectrode for the Diagnosis of Dry Eye Diseases

Electrochemical sensors have garnered significant attention for physiological and clinical applications, particularly in ion detection for diagnostics. Despite advancements in sensor integration, particularly in materials and fabrication approaches, challenges remain in sample collection. Although sensor construction and usage vary widely, common issues persist, especially in the analytical characterization, calibration, and validation of on-body measurements.

Potentiometry, based on ion-selective electrodes (ISEs),¹ is a widely used technique in laboratories due to its versatility, broad dynamic range (3-5 orders of magnitude), fast analysis times, and cost-effectiveness. ISEs convert ion exchange events into voltage signals, with the concentration dependence of the potentiometric signal following the Nernst equation. The ISE calibration plot (EMF versus logarithmic activity) is then used to determine the concentration of unknown ions in samples. ²⁻⁵

Ion Transfer at the Interface between Two Immiscible Electrolyte Solutions (ITIES)⁶ is a relatively new area of research. Fundamental studies have indicated the potential of stripping voltammetric ISEs for the simultaneous detection of multiple analytes in different samples. The successful demonstration of Ion Transfer Stripping Voltammetry (ITSV) and its associated electrode potential as a portable and inexpensive microsensor for real-time sodium ion concentration measurement represents a significant advancement. Tear osmolarity is primarily dependent on sodium ion concentration, which is the main electrolyte present in normal human tear fluid (120-170 mM), rather than potassium (6-42 mM), calcium (0.3-2 mM), or magnesium ions (0.3-1.1 mM). Without wishing to be bound by theory, this innovation can revolutionize osmolarity diagnostics and control.⁷

The innovation described herein comprises an ion-selective voltammetric microsensor designed to measure sodium ion concentration in tears, for example, for diagnosing Dry Eye Disease (DED). Unlike traditional potentiometric sensors that rely on ion-selective electrodes (ISE) or ion-selective field-effect transistors (ISFET) to measure osmolarity under zero current conditions, the approach described herein utilizes ion transfer stripping voltammetry (ITSV).⁶ This technique leverages a conductive polymer membrane, such as poly (3,4-ethylene dioxythiophenc)-poly (styrene sulfonate), PEDOT:PSS, embedded as a solid contact within the sensor. A non-limiting example of the originality and transformative nature of our innovation is the new application of ITSV combined with a conductive polymer-embedded membrane and a sodium-selective ionophore. This combination allows for the direct measurement of sodium ion concentration in tears with improved accuracy and sensitivity. The conductive polymers used in our sensor offer tunable conductivity and biocompatibility, making them ideal for bio-electrochemical systems. Non-limiting, exemplary differences in our innovation from existing work by is the cost-effective, real-time osmolarity monitoring microsensor for DED. The integration of a blend of PEDOT, Polythiophene (PTh), and Polyacetylene (PA)⁸ into a self-adhesive, stretchable dry electrode further enhances the sensor's functionality by serving as a redox intermediary for sodium ions.⁹

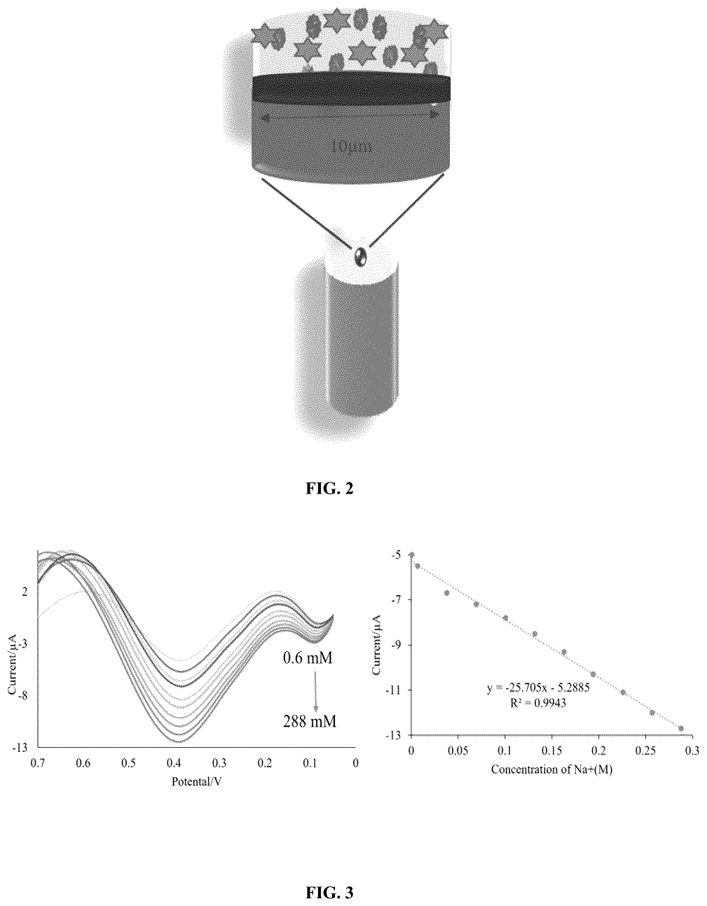

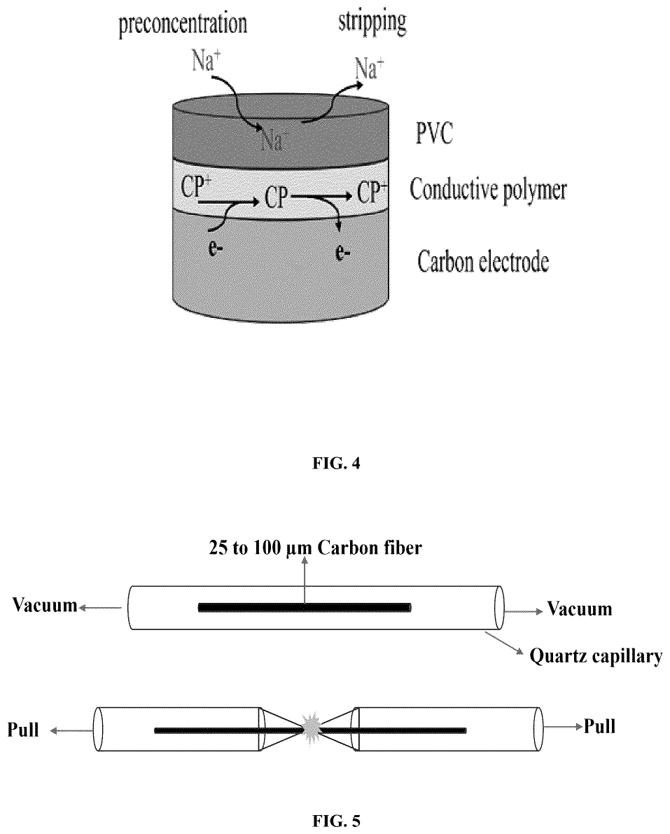

We have dropped cast PEDOT:PSS on a fabricated double polymer 3 mm glassy carbon electrode (GCE) followed by a PVC cocktail containing Na ionophore (IV) (1.54%), Potassium tetrakis-(pentafluorophenyl) borate (KTFAB) (2.07%), 2-nitrophenyl octyl ether (O-NPOE) (67.3%), and PVC (30.5%) in 1 mL of THF (Tetrahydrofuran) to perform ITSV. In embodiments described herein, voltammetric measurements with PVC/PEDOT:PSS modified electrodes were made using a computer controlled CHI660D electrochemical workstation (CH Instruments, Austin, TX). In embodiments, experiments were carried out in a three-electrode cell with a platinum (Pt) counter electrode, an Ag/AgCl reference electrode used as the reference electrode, and a modified glassy carbon electrode (3 mm) as the working electrode in an aqueous solution. In embodiments, sodium ions were pre-concentrated into the PVC membrane by applying 0.05 V for 7 minutes followed by stripping ions from the PVC membrane by linearly sweeping the potential of the GCE toward the positive potential (FIG. 4). At positive potentials, the PEDOT:PSS film becomes oxidized, driving the transfer of Na⁺ ions from the membrane into the aqueous phase. During the potential scan, a peak-shaped current response was recorded in FIG. 2 panel a, illustrating the stripping current proportional to the concentration of Na⁺ ions in the aqueous sample solution (ranging between 0.6 and 288 mM which corresponds to an osmolarity of 1.2 mOsm/L to 576 mOsm/L). The R² value of the regression line fitted to the calibration curve was 0.9943 (FIG. 2 panel b).

Non-limiting, exemplary measurements on sodium show that methods described herein can be successful for sodium detection. We apply sodium (IV) ionophore, highly selective for sodium, to create a membrane selective for sodium ions. Ionophore (IV) is a 16-crown 5-derivative ionophore with a Na⁺-sized cavity that prevents complexation with larger ions such as potassium. The plasticized PVC membrane serves as the reservoir for Na⁺ ions, pre-concentrated from the aqueous sample. The conductive polymer (CP) film (PEDOT: PSS) functions as an ion-to-electron transducer, facilitating continuous current flow between the ionically conductive PVC membrane and the concentrations in sample solutions was applied to determine slope, m, intercept, b, and standard electronically conductive solid electrode, a least-square analysis of peak currents versus Na⁺.¹⁰ In a non-limiting, exemplary experiment, the limit of detection (LOD) for sodium ion was calculated as 0.28 mM (28×10⁻⁵ M) using the innovations described herein. For example, (1) Ion Transfer Stripping Voltammetry (ITSV) incorporating the ionophore (IV) within the outer PVC layer. (2) Ion-selective voltammetric carbon sensor features an electrode with a coated layer of a conductive polymer membrane such as PEDOT:PSS as an embedded solid contact. The breakthrough can be realized through the solution processing of a blend comprising PEDOT:PSS, PTh, and PA, yielding a self-adhesive and stretchable dry electrode. This membrane acts as a redox intermediary for sodium ions. (3) Selective Na⁺ sensors on carbon microelectrodes, utilizing Ion Transfer Stripping Voltammetry (ITSV), with the potential to replace current bulky equipment with a compact and portable system for numerous clinical applications dependent on non-invasive measurements.

### Embedded Solid Contact:

Material Stability—Stability under varying environmental conditions (temperature, humidity) is crucial to avoid degradation and impaired performance. This can be enhanced by using composites resistant to environmental degradation.

Electrical Conductivity—Because inconsistent conductivity can compromise signal stability, we can use materials with consistent performance across diverse conditions.

Solution Processing with PEDOT:PSS, PEDOT, PTh, and PA⁸—in embodiments material formulations can be adjusted to improve material interactions and electrode performance.

Self-adhesive and Stretchable Dry Electrode—in embodiments, the electrode's fatigue resistance can be enhanced by materials or coatings.

Without wishing to be bound by theory, the microsensor described herein can be optimized in terms of scaling costs, sensor stability, membrane selectivity and sensitivity, and membrane durability. Scaling costs-in embodiments, the 3 sensors can be incorporated into a single sensor body. In embodiments, the bill of materials (BOM) for both the sensor and the analyzer can be about $50 or less than about $50. In embodiments, the sensor can withstand low volume tear samples. In embodiments, the ion-selective membrane can exhibit less than about 10% cross-sensitivity to contaminants. For example, unlike potentiometric sensors, which rely solely on the ion-selective membrane for sensitivity, voltametric sensors can also analyze the voltage across the redox potential of the target analyte. In embodiments, the membrane can be durable enough to last at least 6 months.

### Non-Limiting Research and Development

Described herein is a microsensor that integrates ITSV with a specially designed electrode. This innovation combines the ITSV method with a unique electrode configuration featuring a conductive and redox-active polymer layer, enhancing the sensitivity, selectivity, and stability of ion detection. This approach addresses the limitations of conventional electrochemical techniques, particularly in measuring ions that lack redox activity and are challenging to detect.

Non-Limiting Advantages of ITSV comprise: The absence of ion oxidation or reduction, reduced interference and higher sensitivity and selectivity compared to potentiometry, and the inclusion of a programmable voltage generator and current detection circuitry makes it a more sophisticated electrochemical analyzer than current potentiometric and amperometric sensors used in diagnostics.

Traditional voltammetry requires the analyte to be oxidized or reduced at a reasonable voltage (typically less than 2V), which limits its application for ions like sodium. ITSV overcomes this by incorporating a conductive and redox-active polymer between the PVC ionophore layer and the electrode. This design allows for the measurement of ions at low concentrations through a preconcentration stage before the potential is applied. It also ensures high selectivity by using an appropriate window potential for the ion in question.

In embodiments, a two-step technique can be used to achieve high sensitivity. For example, the two-step technique can comprise: (1) Preconcentration: ions are electrolytically concentrated in the polymer membrane, and (2) Releasing: The concentrated ions are dissolved back into the solution for measurement. The process reduces detection limits by two to three orders of magnitude compared to solution-phase voltammetric measurements.

For example, we have applied the ITSV technique to detect nanomolar concentrations of Cr (VI)¹¹ in drinking water and selective serotonin reuptake inhibitors (SSRIs) in environmental water samples,¹² and the pencil lead was modified with an electrochemically deposited 3,4-ethylene dioxythiophene (PEDOT-C14)¹³ indicating the method's versatility and effectiveness.

Described herein, we explore the charge-transport efficiency and stability of an oxidized and reduced poly (3,4-ethylene dioxythiophenc) polystyrene sulfonate (PEDOT: PSS) film, and further employing a blend of PEDOT:PSS, PEDOT, PTh, and PA to create a self-adhesive, stretchable dry electrode, serving as a redox intermediary for sodium ions covered with a sodium ionophore doped PVC membrane for use in a ITSV based microsensor.

For example, ITSV as a method for Na⁺ detection and measurement can be indicated by our Conductive Polymer/PVC Composite Coating/Glass Carbon Electrodes for Selective Detection of Sodium using Ion Transfer Stripping Voltammetry (ITSV) and Enhancing the robustness and sensitivity of the microsensor using iontransfer voltammetry of Sodium-ion by Ion-Selective Stripping Voltammetry.

Without wishing to be bound by theory, the oxidized and reduced conductive polymers film transport charge, and how stable is it under varying conditions?

Without wishing to be bound by theory a blend of PEDOT:PSS, PTh, and PA can create a self-adhesive, stretchable dry electrode that maintains its redox properties and integrates well with a sodium ionophore-doped PVC membrane. For example, a robust and flexible electrode that can withstand mechanical stress while maintaining its electrochemical properties. Successful development of this electrode will demonstrate the potential for scalable production and commercial application.

Without wishing to be bound by theory, the Conductive Polymer/PVC Composite Coating on Glassy Carbon Electrodes can selectively detect sodium ions using lon Transfer Stripping Voltammetry (ITSV).

Without wishing to be bound by theory, the robustness and sensitivity of the microsensor be enhanced for sodium-ion detection using Ion-Selective Stripping Voltammetry.

We can Optimize Conductive Polymer/PVC Composite Coating/Glass Carbon Electrodes for Selective Detection of Sodium using Ion Transfer Stripping Voltammetry (ITSV).

The double polymer layers working electrode consists of a commercial micro carbon electrode, which will be coated with a conductive polymer membrane such as PEDOT: PSS to serve as an embedded solid contact, or a blend consisting of PEDOT:PSS, PTh, and PA will be utilized, resulting in a self-adhesive and stretchable dry electrode. The second layer comprises an optimized plasticized PVC coat containing the first conductive polymer layer containing sodium ionophore.

This objective was achieved by:

Evaluation of electrochemical sensing and activity of CP/PVC composite/Glass Carbon electrodes (GCE) with the ITSV technique to investigate the electrochemical sensing behavior of the modified electrode. As previously explained, applying two layers on the electrode can detect sodium at the micromolar level. The surface area of CPs allows the immobilization of high quantities of PVC composite and, therefore, a higher amount of sodium ionophores, significantly increasing the sensor's sensitivity, and providing a superior electron transfer capability. Most theoretical descriptions of sodium selective membrane potential and selectivity use an equilibrium approach, where the selectivity coefficients of the primary ion over the interfering ion(s) are proportional to the ratio of the corresponding ion-ionophore complex formation constants.

Evaluation of the Selectivity, Reproducibility, and Stability of CP/PVC composite/GCE. Selectivity is an essential feature of a sensor, as high selectivity ensures excellent accuracy. The ITSV technique will investigate some common interfering components found in tears, such as potassium (K⁺), calcium (Ca²⁺), magnesium (Mg²⁺), and proteins like lysozyme and lactoferrin. Known concentrations of these compounds will be tested to determine their potential interference. The repeatability of the modified GCE will be evaluated by conducting several repeat measurements using the same electrode within a single day, employing the ITSV technique to achieve an acceptable relative standard deviation (RSD) value. The CP/PVC composite/GCE will be examined for their storage and operational stability by applying ITSV techniques.

Evaluation of different scan rate responses of the CP/PVC composite/GCE. The diffusion-controlled electrochemical behavior depends on scan rate responses. Different scan rates from 0.01 to 0.10 Vs⁻¹ can be employed to assess the dynamics of heterogeneous electron transfer across the electrode/film layer interface. By increasing the voltage, the sodium concentration at the sensor surface also alters, so a faster voltage sweep causes a more significant concentration gradient near the sensor. The results can indicate the electrode potential against the diffusion of the sodium concentration gradient, indicating that the process is controlled by diffusion and that the electron transfer processes involve freely diffusing sodium ions.

Optimization of the CP/PVC composite/GCE parameters. Voltammetric design choices such as ionophore concentration, PVC film thickness, CP film thickness, and electrode diameter have been chosen arbitrarily. We can fit voltammogram data to models of ITSV and extract parameters that predict the ITSV signal. Then, theoretical models can be applied to fit the data and remove the desired equation unknowns. These parameters can allow us to improve the design parameters to maximize the ITSV signal, minimize the response time, and prolong the longevity. For example, a thinner PVC membrane (app. 200 nm) will increase sodium ion transfer.

The following contributed to the advancement of ion-selective membrane-based sensing technologies by optimizing membrane composition and solid contact types, as well as leveraging nanoparticles and other parameters to enhance electrode performance.
1. Optimization of Membrane Composition and Solid Contact Type

Identification of the most effective plasticizers and sodium ionophores for enhancing sensitivity and selectivity of the ion-selective membrane (ISM) composite.

Characterization of the optimal membrane composition in terms of sensitivity, selectivity, and stability.

Establishment of a comprehensive understanding of the effects of different membrane components on the performance of ISM composites.
2. Utilization of Nanoparticles and Conductive polymers

Integration of embedded solid contacts or blends comprising PEDOT:PSS, PTh, and PA to enhance electrode activity.

Enhancement of electrode performance through the incorporation of nanoparticles “Gold Nanoparticles, Graphen” leading to improved conductivity and sensing capabilities. Without wishing to be bound by theory the gold nanoparticles can possess exceptional electrical conductivity, which enhances the overall electron transfer rate at the electrode surface. This leads to faster and more accurate signal transduction. Additionally, the nanoscale structure of AuNPs can provide a high surface-to-volume ratio, increasing the active surface area available for interaction with analytes. AuNPs are biocompatible, making them ideal for biosensors and applications where biological molecules (e.g., proteins, enzymes, or DNA) need to be immobilized.

Moreover, Gold nanoparticles exhibit excellent chemical stability, ensuring long-term performance and durability of the electrode. Graphene, a single layer of carbon atoms arranged in a hexagonal lattice, exhibits extraordinary electrical conductivity due to its high charge-carrier mobility. This facilitates efficient electron transfer during sensing.

Mechanical Strength and Flexibility: Graphene is both strong and flexible, enabling the fabrication of durable and versatile electrodes, even for wearable or flexible devices.

High Surface Area: Similar to AuNPs, graphene provides an extensive surface area, enhancing the adsorption and interaction of analytes with the electrode.

Electrochemical Properties: Graphene's unique electrochemical behavior improves the sensitivity of the electrode, allowing for the detection of very low concentrations of analytes.

We can enhance the robustness and sensitivity of the microsensor using ion transfer voltammetry of Sodium-ion by Ion-Selective Stripping Voltammetry.

Development of a Sensitive, Rapid, and Cost-Efficient Microelectrode Using ITSV to Determine Sodium Ions in Tears. Herein, we describe the development of a highly sensitive, rapid, and affordable microelectrode for detecting sodium ions in tears. An exemplary method employed is Ion-Selective Stripping Voltammetry (ITSV), which enhances the sensitivity and selectivity of sodium ion detection, particularly in low concentrations found in tear samples.

### Non-Limiting, Exemplary Developments:

### Fabrication of Sodium Selective Microelectrodes (SSMEs) Using ITSV

Herein, we describe carbon-based microelectrodes (CMEs) that are designed to detect sodium ions in tear samples. ITSV can be used to optimize the selective transfer of sodium ions across a membrane, ensuring that the microelectrodes are highly sensitive and capable of detecting even low concentrations of sodium.

### Development of Conductive Polymers (CPs)/CME Composites

To improve the performance of the CMEs, conductive polymers (CPs) can be integrated with the microelectrodes. The CP/CME composites can enhance the conductivity and robustness of the sensor, making it more reliable and efficient for rapid sodium ion detection in tears.

### Without Wishing to be Bound by Theory, We Can use Anti-Fouling Materials

To combat sensor fouling, where unwanted materials from tear samples accumulate on the sensor and reduce performance, anti-fouling materials will be applied. These materials will prevent contaminants from adhering to the sensor surface, ensuring that the sensor can accurately detect sodium ions. The sensor's selectivity will be tested using artificial tears to verify its effectiveness in detecting sodium without interference from other tear components. Strategies such as functionalizing the interface with materials like polyethylene glycol (PEG) or zwitterionic species will help maintain a hydrated sensor surface, minimizing fouling.¹⁴

### Evaluation of Sensor Membrane Reproducibility

We can develop a sensor membrane that consistently produces reliable and accurate results across multiple samples for sodium ion detection.

### Determination of Sensor Lifetime and Membrane Analysis

We can assess the durability of the sensor by performing repeated measurements until the membrane begins to degrade and the detection limits are affected. Scanning Electron Microscopy (SEM) can be used to analyze the membrane's thickness and composition.

We have fabricated the electrode design depicted in FIG. 5. In our laboratory, we have the advantage of the P-1000 Micropipette Puller, a versatile tool capable of crafting customized pipettes for diverse applications. To streamline this process, we can integrate a programmable control system into the P-1000, enhancing our ability to manipulate pulling parameters. By utilizing gold fibers and integrating programmable control, we can achieve finer control over the pulling process and improve reproducibility. This control system enables us to design application-specific pipettes from a broad spectrum of glass compositions and sizes, facilitating precise experimentation and analysis. Different programs will be developed to identify the optimal parameters for manufacturing gold microelectrodes.

FIG. 6 panel a illustrates the preparation of microelectrodes with a laser puller. The carbon fiber is inserted into the glass capillary and positioned at its midpoint by gently tapping on the bench. Vacuum is applied at both ends of the capillary to aid in the pulling process, and a specific program is selected in the laser puller to fabricate the quartz/gold assembly with a fine tip.

Without wishing to be bound by theory, we can enhance the sensor membrane using antifouling techniques to maximize sensitivity and selectivity while minimizing fouling. Sodium ions will be detected in both artificial and real tear samples.

### Non-Limiting Impacts

The Motivation: Dry eye disease (DED) is one of the most common reasons for eye doctor visits, with causes including autoimmunity, hormonal imbalance, dehydration, and environmental factors. Current diagnostic methods—such as tear breakup time, Schirmer test, and tear osmolarity—have limitations, including subjective interpretation, result variability, and incomplete assessment of tear film composition. A new ion-selective microelectrode (FIG. 4) can offer more precise, objective measurements of tear film stability and composition, addressing these issues by providing consistent, quantifiable data on tear ion content. While there is no single gold standard for diagnosing DED, elevated tear osmolarity is a key indicator, with hyperosmolarity associated with disease severity. Hyperosmolarity can damage corneal cells and cause refractive instability, potentially leading to vision loss. Existing methods for measuring tear osmolarities face challenges, such as the need for microliter tear samples, specialized expertise, continuous equipment maintenance, and high operational costs. The new microsensor described herein uses innovative ion transfer stripping voltammetry (ITSV) to detect tear osmolarity, enabling rapid, near-real-time measurements. The microsensor described herein can enhance DED diagnosis, treatment monitoring, and research, leading to personalized care strategies.

### Societal Benefit

Enhancing Public Health and Well-being. The development of affordable, mass-produced sensors for diagnosing dry eye disease (DED) represents a significant advancement in public health. DED affects millions of individuals, causing discomfort and potentially leading to more severe health issues if not diagnosed early. Currently, diagnostic methods for DED can be expensive and often require access to specialized medical facilities. By creating a portable clinical analyzer that provides fast, accurate, and lab-quality results within minutes, this project addresses a critical gap in healthcare. Such technology can enable widespread early-warning diagnoses, allowing for timely treatment and management of DED. This proactive approach not only improves patient outcomes but also reduces the burden on healthcare systems, leading to lower overall healthcare costs and enhanced quality of life for affected individuals.

The microsensor described herein provides significant economic advantages. By enabling large-scale, low-cost production, this technology addresses a pressing market need for effective, non-invasive diagnostic tools in the medical field.

In addition to its direct health and economic benefits, the development of this microsensor technology will have a profound impact on communities. Improved access to accurate and affordable diagnostics will empower individuals to seek timely medical attention, leading to better management of Dry Eye Disease (DED) and a reduction in the associated discomfort and complications. By making advanced diagnostic tools more widely available, the project helps bridge gaps in healthcare access, particularly in underserved or remote areas. Enhanced diagnostic capabilities will support community health initiatives, reduce the strain on healthcare providers, and foster a more informed and proactive approach to managing eye health. This ripple effect of improved health outcomes and quality of life underscores the broader societal benefits of the proposed technology, making it an asset to communities across diverse regions.

The microsensor described herein can be used for Dry Eye Disease (DED) diagnosis, a condition affecting approximately 25% of ophthalmology patients. DED is more prevalent among older adults, women, and individuals with autoimmune conditions. The costs associated with managing DED are significant, with annual U.S. expenditures ranging from $3 billion to $4 billion. Existing diagnostic methods, such as Schirmer's test¹⁸ and Tear Lab,¹⁹ is often costly, time-consuming, and uncomfortable for patients. Clinics and patients seek faster, more accurate, and affordable diagnostic alternatives. The high-speed, low-cost microsensor we propose addresses these needs by providing efficient and less invasive diagnostic solutions for DED, reducing the risk of complications, and offering an attractive value proposition for clinics and patients alike.

The device described herein can be useful for optometrists, ophthalmologists, and medical device companies specializing in medical sensors. The Electrochemical Medical Sensors market, which was valued at $26.8 million in 2019 and represented 71% of the market in 2021, is expected to grow at a rate of 6.65% by 2030. Additionally, the broader medical sensors market, which reached $13.7 20 billion in 2022, is projected to grow to $25.8 billion by 2030, at a rate of 7.33%. 21 These trends underscore the significant opportunity for our technology in a growing market.

Our market validation includes insights from clinics that express dissatisfaction with current DED diagnostic methods due to their cost and variability. The microsensor's ability to provide accurate results at a fraction of the cost makes it a compelling alternative. With projected costs of less than $50 per electrode, our product presents a cost-effective option compared to devices like Tear Lab ($10,800 per device) and I-PEN,²² with their variable accuracy.

Patient and Provider Feedback. Initial feedback from both patients and healthcare providers has been overwhelmingly positive. Patients appreciate the non-invasive nature of the microsensor and its potential for more frequent monitoring, which aligns with their desire for more accessible and less uncomfortable testing options. Providers highlight the potential for the microsensor to streamline diagnostic processes and improve the efficiency of patient care. This feedback underscores the microsensor's potential to fill a significant gap in the market, offering a practical, reliable solution that addresses both the cost and accuracy concerns prevalent in current DED diagnostic practices. By conducting early detection and precise monitoring of DED, we position the microsensor as a game-changer for clinics looking to enhance patient outcomes while reducing expenses.

The sensor described herein can be used by clinics that lack advanced diagnostic tools to generate revenue, enabling them to offer DED diagnoses using our microsensor. The low cost of the microsensor allows for significant cost savings and new revenue streams for clinics by billing for DED diagnostic services.

Our device offers significant cost benefits by reducing operational and maintenance costs, enhancing diagnostic accuracy, and saving time with rapid, real-time measurements. Although the initial investment may be higher, its efficiency and durability provide long-term cost savings and improve overall healthcare value.

Our microsensor provides a faster, more accurate, and cost-effective solution for diagnosing Dry Eye Disease. Unlike existing technologies that are costly and prone to variability, our solution offers clinics a tool that delivers reliable results while significantly reducing operational costs. Patients will benefit from earlier diagnosis, leading to improved treatment outcomes, and clinics can offer high-quality care at lower costs.

The current DED diagnostic market is dominated by established players like Tear Lab and I-PEN. However, their products are hindered by high costs and inconsistent results. The microsensor described herein offers a clear competitive advantage with its combination of affordability, accuracy, and case of use. Our strategy includes leveraging these advantages to penetrate the market and build strong relationships with healthcare providers. Additionally, our approach involves targeting underserved clinics and regions where existing diagnostic tools are either unavailable or prohibitively expensive. By offering a cost-effective, reliable alternative, we aim to disrupt the market, enhance patient access to quality care, and position ourselves as a leader in innovative DED diagnostics. Our ongoing market analysis and adaptation to emerging trends will ensure that we maintain a competitive edge and meet evolving healthcare needs effectively.

### REFERENCES CITED

1. Gao, L.; Tian, Y.; Gao, W.; Xu, G. Recent Developments and Challenges in Solid-Contact Ion-Selective Electrodes. Sensors 2024, 24, 4289.
2. Abbaspour A.; Izadyar A. Multi-Wall Carbon Nanotube Composite Coated Platinum Electrode as a Sensitive Sensor for Detection of Cr (III) in Natural Waters. Anal Bioanal Chem. 2006 386:1559-1565.
3. Bobacka J., Ivaska A., Lewenstam A. Potentiometric ion sensors. Chem. Rev. 2008, 108:329-351.
4. Bakker E.; Pretsch E. Modern Potentiometry. Angew. Chem. Int. Ed. 2007, 46:5660-5668.
5. Gao W.; Emaminejad S.; Nyein H. Y. Y.; Challa S.; Chen K.; Peck A. Fully integrated wearable sensor arrays for multiplexed in situ perspiration analysis. Nature. 2016, 529:509-14.
6. Izadyar A. Stripping Voltammetry at the Interface between Two Immiscible Electrolyte Solutions: A Review Paper. Electroanalysis 2018, 30:2210-2221.
7. Bron, A. J.; Willshire, C. Tear Osmolarity in the Diagnosis of Systemic Dehydration and Dry Eye Disease. Diagnostics 2021, 11, 387.
Georgiev, G. A.; Yokoi, N. Editorial of Special Issue “Differential Diagnosis for Dry Eye”. Diagnostics 2021, 11, 910.
8. D. C. Harris, Quantitative Chemical Analysis, 8th ed., W.H. Freeman and Company, New York, 2010, pp. 85-105.
9. Pan, Y.; Zhang, J.; Guo, X.; Li, Y.; Li, L.; Pan, L. Recent Advances in Conductive Polymers-Based Electrochemical Sensors for Biomedical and Environmental Applications. Polymers 2024, 16, 1597.
10. Izadyar A.; Al-Amoody F.; Ranawaka Arachchige D. Ion transfer stripping voltammetry to detect Nanomolar concentrations of Cr(VI) in drinking water. J. Electroanal. Chem. 2016, 782:43-49.
11. Izadyar A.; Ranawaka Arachchige D.; Cornwell H.; Hershberger J. L. Ion Transfer Stripping Voltammetry for the Detection of Nanomolar Levels of Fluoxetine, Citalopram, and Sertraline in Tap and River Water Samples. Sens. Actuators B Chem. 2016, 223,226-233.
12. Izadyar A, Kim Y, Ward Muscatello M M, Amemiya S. Double-Polymer-Modified Pencil Lead for Stripping Voltammetry of Perchlorate in Drinking Water. Journal of Chemical Education. 2012, 89:1323-1326.
13. Izadyar A.; Hershberger J. C.; Robert R. Voltammetric Assessment of Ions Transfer at Ionophore-Graphene Based Polymeric Membranes. Electroanalysis 2018, 30:2580-2583.
14. Izadyar A., Van M. V., Miranda M., Weatherford S., Hood E. E., Electrocatalytic Effect of Recombinant Mn Peroxidase from Corn on Microbiosensors to Detect Glucose, Biocatal. Agric. Biotechnol. 2022, 43:102445.
15. Izadyar A.; Van M. V.; Miranda M.; Weatherford S.; Hood E. E. Development of a Highly Sensitive Glucose Nanocomposite Biosensor Based on Recombinant Enzyme from Corn. J. Sci. Food Agric. 2022, 102:6530-6538.
16. Wang X.; Fan X.; Wu Y.; Mou Y.; Min J.; Jin X. Rear 4-min Schirmer test, a modified indicator of Schirmer test in diagnosing dry eye. Sci Rep. 2022 15:6272.
17. Yoon D.; Gadaria-Rathod N.; Oh C.; Asbell P. A. Precision and accuracy of TearLab osmometer in measuring osmolarity of salt solutions. J Current eye research. 2014 39:1247-1250.
18. Fagchi R.; Al-Bishry A. B.; Alanazi M. A.; Abusharha A.; El-Hiti G. A.; Masmali A. M. Investigation of the repeatability of tear osmolarity using an I-PEN osmolarity device. Taiwan J Ophthalmol. 2020 11:168-74.
19. Shimazaki J.; Sakata M.; Den S.; Iwasaki M.; Toda I. Tear film osmolarity measurement in Japanese dry eye patients using a handheld osmolarity system. Diagnostics. 2020 10:789.
20. Park J.; Choi Y.; Han G.; Shin E.; Han J.; Chung T. Y. Evaluation of tear osmolarity measured by I-Pen osmolarity system in patients with dry eye. Sci Rep. 2021 11:7726.
21. Tavakoli A.; Markoulli M.; Flanagan J.; Papas E. The validity of point-of-care tear film osmometers in the diagnosis of dry eye. Ophthalmic Physiol Opt. 2021 10:1111.
22. König S.; Priglinger S.; Schaumberger M.; Messmer E. M. Tear Film Osmolarity in Normal Individuals: Comparison of Two Osmometers. Klin Monbl Augenheilkd 2020 237:649-54.
23. Dana R.; Meunier J.; Markowitz J. T.; Joseph C.; Siffel C. Patient-Reported Burden of Dry Eye Disease in the United States: Results of an Online Cross-Sectional Survey. Am J Ophthalmol, 2020, 216, 7-17.
24. Verified market research, Medical Sensors Market Size and Forecast, Pharma & Healthcare. (2022).

### Example 3

### Example 3: A Disposable Electrochemical Sensor for the Diagnosis of Dry Eye Disease.

Water must function properly in various organs as a vital body element. The body's response to fluid loss is to conserve what remains in the body. This causes symptoms of dehydration such as thirst and dry mouth, muscle cramps, headache, lightheadedness, sleepiness, and lack of tear production. When the eyes stop producing tears, it leads to a lack of proper lubrication. This can lead to dry eyes, eye fatigue, and vision problems. Normal osmolarity is essential for adequate production, maintenance, and balanced tear restriction.¹³ The prevalence of dry eye in the U.S. is increasing yearly. ^{14,15} The global epidemic with an aging population is an essential factor in this growth. Various factors, including autoimmunity, hormonal imbalance, dehydration, and a harmful environment, can cause DED.¹⁶ Many systemic diseases have symptoms related to dry eyes, such as diabetes mellitus (D.M.), rheumatoid arthritis (R.A.), multiple sclerosis (M.S.), and Sjögren's syndrome (S.S.).¹⁷⁻¹ Advances in understanding dry eye will lead to better, more rapid treatments for the millions of people in the United States affected by this disease. Our proposed high-speed, low-cost sensor for diagnosing this disease can prevent the need for lengthy and complex treatments and eye infections. This disease is rarely diagnosed due to limitations such as microliter tear samples, the need for specialist expertise, continuous equipment maintenance, and the operation's duration and cost.^{20,21} Failure to quickly measure dry eye can affect the quality of vision, lead to the darkening of the stroma, and cause pain. Tear dysfunction is one of the most common causes of corneal epithelial disease. In some embodiments, a screen-printed sensor described herein can assess tear osmolarity quantitatively using about 20 μL.

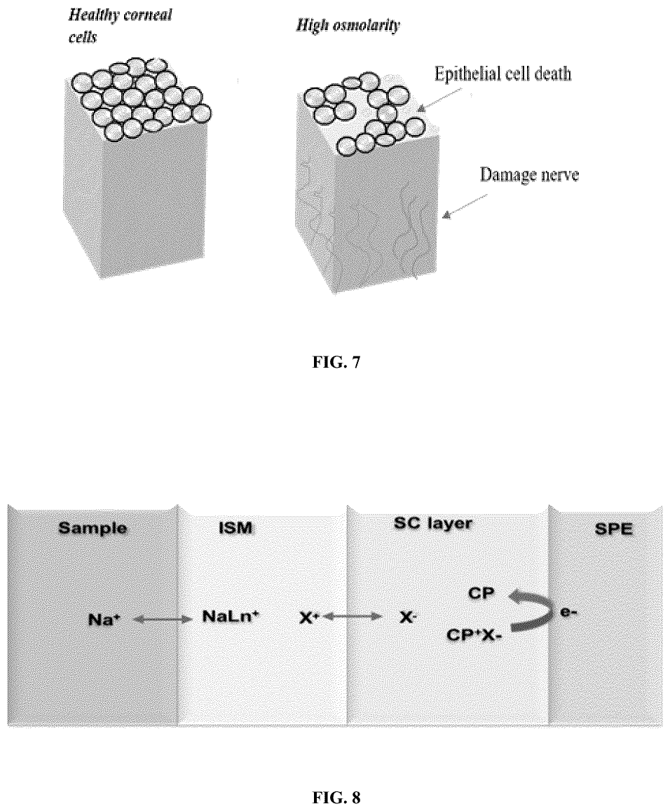

In addition, our sensor will not require unique training and will be a small, portable device. Lowering the cost and increasing the portability of dry eye symptom monitoring makes it easier to keep up-to-date measurements for individual patients. Additionally, if osmolarity is outside acceptable parameters, ophthalmologists can act more quickly to provide treatment. Hyperosmolarity damages corneal cells (FIG. 7) and causes refractive instability. Surgical intervention may not be necessary for patients who receive timely DED diagnoses. Additionally, if osmolarity is outside acceptable parameters, ophthalmologists can act more quickly to provide quicker treatment. Regular screening for DED using our proposed sensor will ensure that individuals with DED diagnoses gather the information needed to receive more effective and efficient patient care. Ion-sensitive electrodes (ISEs) as electrochemical sensors²²⁻²⁶ have shown exemplary performance in ion selection, with low detection limit and fast response.

ISEs are widely used due to their advantages of simple fabrication, low cost, and vital portability. They have been used for years to detect essential ions in the body's bloodstream, urine, and other fluids. For this reason, they are an essential part of the ion monitoring devices in the market. ISEs are commonly used in the potentiometry technique, where the open circuit potential records against a reference electrode under zero current conditions. The electrode potential changes linearly with the logarithm of the activity of the target ions in the sample in the Nernst response region. ISEs are advantageous because of their versatility and high selectivity due to various ionophores. ^{3,27} The voltammetric measurement mode allows for multiple ion detection, and the ISE is used to quantify multiple ions with higher sensitivity than potentiometry. When the ionophore is discharged entirely, the primary ion exchanges with the desired secondary ion at higher potentials; as a result, Na⁺ ion peaks can be obtained with ISE containing Na⁺ selective ionophore and at more positive ISE potentials peaks related to other ions.

Described herein we can apply modified screen-printed electrodes (SPEs) containing ionophores using ion transfer stripping voltammetry (ITSV) to detect concentrations of Na⁺ in tears at the osmolarity range. We will use several effective materials to develop our sensors, such as conductive polymers, various carbon materials, nanomaterials (carbon nanotubes, nanowires, nanoparticles, and quantum dots (QDs)²⁸, and molecular redox couples.²⁹ Poly (3,4-ethylene dioxythiophenc) (PEDOT) is a promising conducting polymer due to its extraordinary electrical properties, long-term stability, and biocompatibility. In addition, PEDOT doped with poly (styrene sulfonate) (PSS) (PEDOT/PSS)¹² has recently been widely used in sensors for bioengineering and medical applications. In our non-limiting, exemplary studies, we used PEDOT/PSS to fabricate our sensor, and the results indicate that the effectiveness of this compound as a solid contact.

The global ophthalmic diagnostic devices market stood at USD 2.41 billion in 2018 and is projected to reach USD 3.86 billion by 2026, exhibiting a CAGR of 6.1% during the forecast period^{14,15}, and the market size for electrochemical medical sensors was 26.8M USD in 2019, accounting for 71% of the market in 2021. The electrochemical sensors market is expected to grow by 6.65% in 2030.²⁵ Meanwhile, the medical sensors market size was $13.7B in 2022 and is projected to reach $25.8B by 2030, growing at a rate of 7.33%. ²⁶

There are specific challenges to diagnosing this disease. The Schirmer test 30 is widely used to diagnose dry eye but has many disadvantages, including poor reproducibility, low sensitivity and specificity, and severe patient discomfort. A Tear Lab test is also available, which is less common due to its high-cost device, about 10K; each test card costs $14.00 per eye and only available in a few clinics and hospitals. Abnormal body fluids are an essential diagnostic information source in clinical applications. Solid Contact Ion-Sensitive electrodes (SC-ISE) are virtual devices that monitor a patient's health status using body fluids and blood tests. SPEs are cost-effective, easy to use, and the key to fast and efficient data collection. In addition, they represent long-term and real-time monitoring and efficient devices for rapid measurement in one-step without fear of contamination. Diagnosing dry eye using physiological biomarkers such as gene technology (to assess for rheumatoid arthritis or thyroid problems that can cause DED) is challenging due to complex processing for diagnosis and poor correlation between clinical signs and symptoms. Therefore, the measurement of tear osmolarity using electrochemical sensors as a direct, rapid, and accurate method for DED is crucial. In embodiments, the microsensor described herein aligns with the National Eye Institute (NEI)'s NOSI for diagnosing dry eye disease (NOT-EY-21-007) for Anterior Segment Initiative to identify and develop new biomarkers and effective methods to diagnose DED.

A non-limiting, exemplary embodiment of the innovation is the use of ITIES to diagnose dry eye. This technique is very new compared to the usual methods of voltammetry and potentiometry because: (1) Priority compared with voltammetry because ions are not readily oxidized or reduced and (2) The interfering effect and low sensitivity of the potentiometric methods are improved using ITIES.

Amperometric or voltammetric sensors add a layer of selectivity by adjusting the voltage of a measuring electrode to the oxidation or reduction potential of an analyte ion of interest—if the analyte can be oxidized or reduced. ITSV, which works based on ion transfer, acts critically when the target ion is not easily reduced or oxidized. The desired ion is absorbed in the ion-selective membrane, and an active and redox-conducting organic compound moves in the inner membrane. In this case, with sodium ion as a cation, the poly (3,4-ethylene dioxythiophenc) polystyrene sulfonate (PEDOT) as an organic compound is reduced at the electrode to maintain charge neutrality. We⁵⁻¹¹ have successfully investigated ITSV on several ions. These include antidepressants, lead, and chromium (VI) have worked successfully. We apply sodium ionophore (IV), highly selective for sodium, to create a selective membrane for sodium ions. Ionophore (IV) is a 16-crown-5 derivatives ionophore with a cavity the size of Na⁺, which prevents the formation of complexes with larger ions such as potassium. Evaluation of the relationship between ionophore chemical structures and ion selectivity properties can help host-guest chemistry develop a highly selective ionophore for analytes.

In non-limiting, exemplary embodiments, the sensor described herein can use hydrophobic conductors with large surface area and high double-layer capacity (use of nanostructured materials) which offers excellent potential stability for improving SC-ISE.³¹ Many ISEs have been developed over the years; their designs have recently changed from bulk electrodes with an aqueous internal filling solution to planarable solid contact electrodes. However, despite the use of planarable electrodes, there are limitations such as sensor lifetime (the elapsed time from the beginning of sensor operation until the sensor activity dies), which is due to the weak stability of the membrane. If solid sensor contact membranes are not mechanically fixed in place, the membranes will delaminate and separate from the underlying solid, rendering the sensor unusable. Even slight external mechanical stress or thermal expansion can lead to membrane delamination and, thus, device failure. Described herein, we use solid contacts, which are a means of miniaturizing the electrode. In addition, using SC as operating parameters for ion-selective polymer membranes based on ionophores can create the surface balance for the membrane and confer the least potential drifts. SPEs³², modified in various ways, are allow creativity in sensor fabrication. SPEs are considered one of the best electrodes for in situ analysis in clinical applications are those with fast response, design flexibility, small sample size, excellent sensitivity, and absence of pretreatment processes. Despite the high correlation between tear osmolarity and eye abnormalities, several methods to detect it reply on expensive osmometers.³⁻³⁵

### Non-Limiting Experiments

Potentiometry measurement using the open circuit potentiometry (OCP) technique was studied. The response of the SC-SP-ISE with thin membranes is illustrated in FIG. 1 panel (A), where the measurement procedure consisted of a solution in a range of 2.5×10⁻⁶ to 4.8×10⁻¹M NaCl in a phosphate buffer solution pH 7.0(PBS). FIG. 1 panel (A) depicts the OCP characteristics of one of the electrodes and depicts the increase in the potential as the developed Na⁺-SC-SP-ISE displays a wide linear sensing range of 3.37×10⁻⁵ to 2.40×10⁻¹ mol/L or 6.74×10⁻⁵ to 4.80×10⁻¹ Osm/L. We applied thirteen individual sensors with identical composition, and the results showed a near Nernstian slope of 57.2±1.35 (n=13) and a lower limit of detection of 0.026±0.031 mM, with linear correlation R²=0.9961±0.002 (n=13). FIG. 1 panel (B) Shows a correlation coefficient for one of the measurements. Our studies indicate that the modified SPE using the OCP method can be used for detecting Na⁺ and can be applied as a simple device for monitoring tear osmolarity.

Constructing the SC-SP-ISE. We applied a carbon screen-printed electrode coated with two layers, PEDOT:PSS layer drop cast directly onto the carbon surface and allowed to dry at room temperature. Subsequently, the ion-sensing membrane (ISM) composite was drop-cast evenly onto the same surface of the electrode. The ISM composite was prepared using a mixture of polyvinyl chloride (PVC), solvent mediators, lipophilic sodium tetrakis (4-fluorophenyl) borate dehydrate Na-ionophore, and tetrahydrofuran (THF). The mixture was sonicated for 30 min before use. After each coating, the membrane was air-dried for 12 h. SPEs were then conditioned for 60 min in a 10⁻³M solution of NaCl.

Assembling the Electrochemical Cell. Computer-controlled CHI660D electrochemical (CH Instruments, Austin, TX) Analyzers/Workstations are designed for general-purpose electrochemical measurements. The system contains a fast digital function generator, high-speed data acquisition circuitry, potentiostat, and a galvanostat. We used SPE, which is commercially available (Metrohm, Drop Send). The reference electrode and counter electrode were made of silver and carbon, and the working electrode was made of carbon (4 mm diameter).

### Screen-Printed Electrode (SPE) for Selective Diagnosis of Sodium Using Open Circuit Potential (OCP) Potentiometry

The SC-ISE described herein can meet the need for an accessible and flexible solution through its portable and disposable nature, ability to yield reproducible outcomes, and low price. A non-limiting advantage of the sensor described herein is its application of voltammetric ISEs, wherein an ion is transferred from the aqueous solution to the membrane or vice versa, charge neutrality is maintained by the corresponding oxidation or reduction of conductive polymers (CPs). In this case, the membrane does not need electroactive species. Therefore, sodium ion as a non-electroactive species measurement is possible. Described herein are solid contact ion selective electrodes (SC-ISEs), which are of great interest, especially for real-time and non-invasive analysis of ions in biological fluids. SC-ISEs (FIG. 8) have revolutionized with improved stability and reproducibility in actual samples. Introducing new materials can significantly facilitate the understanding theoretical potentiometry and wearable applications of SC-ISE.

In the electrode structure of the most advanced SC-ISE standard model, an ion-to-electron converter layer can transfer the ion concentration to the electron signal and stabilize the potential at the substrate/ion selective membrane (ISM) interface. Using nanoparticles (NPs) to modify screen-printed electrodes (SPEs) can increase the mass transfer rate, electrocatalytic activity, and a higher reactive surface. We can use drop casting, a simple technique to modify the surface of an SPE. The solid contact will include particles of nanotubes or nanoparticles and a conductive polymer such as PEDOT:PSS, which experimentally shows maximum conductivity. To improve the thickness of this layer, we can use the spin coating method. The spin coating allows the production of a uniform distribution of nanoparticles on the surface of the screen-printed electrode, and the speed of rotation of the thickness of the deposited layer can be controlled. Scanning electron microscopy (SEM) analyzes the morphology of the modified electrodes to determine the sensor activity at each optimization step. Described herein are solid-supported ISEs/SPE to explore the advantages of ion transfer voltammetry to conduct selective and sensitive detection of Na⁺. A thin ion-selective polymer membrane supported by a solid electrode allows the formation of a stable and conductive liquid/liquid system. In embodiments, an intermediate layer for conjugated conducting polymers and nanomaterials can facilitate charge transfer between an ionically conducting membrane and an electronically conducting solid.

The approach will use modified solid contact screen printed-ion-selective electrodes (SC-SP-ISE) to detect Na⁺. SC-SP-ISE is a technique in potentiometric analysis with many advantages related to small electrode areas. These advantages include, but are not limited to, small thickness, high-temperature tolerance, cost-effectiveness, sensitivity, selectivity, robustness, and disposability. In embodiments, the long-term stability of the response can be improved by the presence of highly hydrophobic conducting polymers such as PEDOT:PSS or a derivative thereof. The results of this study will have potential applications in dry eye assays. In addition, conductive polymeric nanomaterials, and ionophores can be applied to improve the proposed sensor.

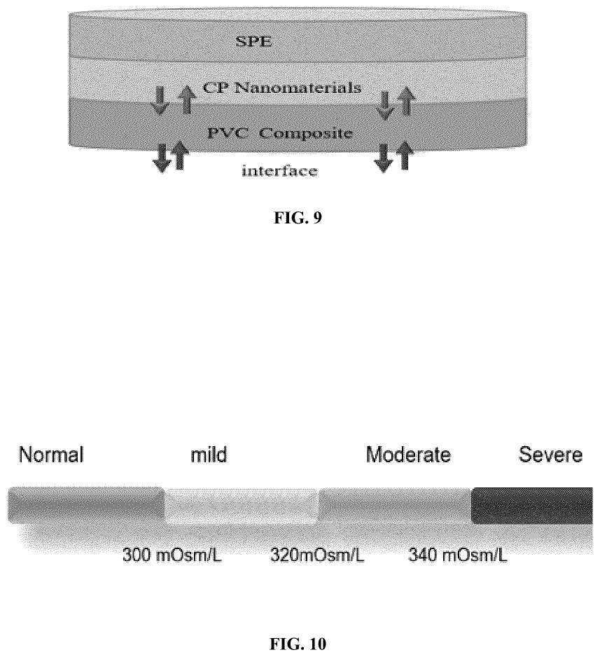

These nanomaterials and ionophores confer excellent conductivity, high catalytic activity, selectivity, an increased number of active sites on the surface, and good adsorption of the analyte compound. We obtained results indicating that SPE using the OCP technique can measure sodium in the tear osmolarity range. We can add nanomaterials to the solid contact layer. Without wishing to be bound by theory, the small size of the particles can promote a large surface area, high electrical capacity of the electrodes, improved concentration range, and SC stability. Most theoretical descriptions of ISE membrane potential and selectivity use an equilibrium approach (FIG. 9), in which the selectivity coefficients of the primary ion over the interfering ion(s) are proportional to the ratio of the formation constants of the corresponding ion-ionophore complex. When performing a potentiometric measurement, the sample usually experiences low disturbance. Measurements in ISEs are not affected by sample color or turbidity, making them suitable for various applications. Cheaper and faster approaches enable ophthalmologists to diagnose dry eye earlier and provide treatment options more quickly.

Without wishing to be bound by theory the membrane composition and solid contact type can use various types and amounts of plasticizers as known in the art and a variety of sodium ionophores. The molar ratio of ionophore/Sodium tetrakis-[3,5-bis(trifluoromethyl) phenyl]borate (NaTFPB) can also be varied.

We can enhance the robustness and sensitivity of the sensor using ion transfer voltammetry at the interface between two immiscible electrolyte solutions (ITIES) Sodium-ion Detection by Ion-Selective Stripping Voltammetry. In embodiments, we can obtain the electrochemical measurement of sodium using the ion transfer voltammetry method at the interface between aqueous solutions and organic electrolytes. We can enhance our ion sensing capabilities by developing voltammetric ion selective electrodes (ISE) based on active ion transport at the liquid/liquid interface (FIG. 3). The voltammetric measurement of biological and environmental media is challenging due to the mechanical instability and high resistance of the conventional liquid/liquid system.

Traditionally, these limitations are circumvented through zero-current equilibrium potentiometry with ISE-based robust polymer membranes. However, ion transfer voltammetry at the interface between two immiscible electrolyte solutions (ITIES)⁵ leads to an electrochemical phenomenon that enables a susceptible technique for detection in the wide range, which is helpful for environmental, industrial, food safety and medical applications as well as for biomedical diagnoses. In this method, a thin PVC membrane supported by a solid electrode is an emerging approach to facilitate ion transfer voltammetric sensor applications. A small ohmic potential drop across the thin membrane enables voltammetric measurements at a macroscopic interface at the electrode surface. We can also investigate membrane fouling which is a factor in their practical application. Once a biofilm has formed on the sensor surface, it is challenging to remove it unless harsh and destructive chemical or physical treatments are used. Therefore, it can be more practical to focus on preventing fouling before biofilm formation. One method for fouling prevention is to use chemical modifiers that increase hydrophilicity by using polar chemical groups, hydrophilic polymers, and self-assembled layers. Examples of these chemical modifiers include polyethylene glycol (PEG), its derivatives, and other polymers such as polysaccharides, polyoxazolines, and poly (hydroxyacrylates), which all have antifouling properties.

Perform a Comparative Analysis to Identify Effective Sensor Materials. The interaction facilitates between the material and the analytes with a higher surface area of the electrode, leading to high sensitivity. The small dimensions enable fast adsorption/desorption kinetics for analytes in the material, resulting in fast response times. Conductive polymeric nanomaterials also have a solid potential to produce sensory performance compared to individual use. Studies indicated that PEDOT with a highly conductive state (more than 3000 S cm⁻¹ under specific conditions doped with PSS improves adhesion and possibly long-term sensor survival. Indeed, PEDOT-PSS. is recognized as an improved electronic interface for a wide range of applications.

Graphene (GO) Mixed with PEDOT:PSS: Graphene is one of the newest materials added to the carbon allotrope family. This single layer of atoms arranged in a honeycomb network includes high mechanical properties and significant electrical conductivity. Therefore, graphene can be a candidate for various applications, such as sensors. We will apply PEDOT:PSS/GO to increase the mechanical properties and strengthen the sensor substrate. SC-SP-ISE has a more active surface and has the advantages of being biocompatible and sensitive and providing selectivity, and stability using PEDOT:PSS/GO as solid contact on SPE.

Polyion Transfer at Polarizable Biocompatible Membrane/sample Interface. An issue for clinical and biological applications of chemical sensors is biofouling. We can investigate biocompatible polymer membranes to protect polyion transport from adsorption interferents in tears. PVC membranes are sufficiently biocompatible for small ion potentiometry.

Membrane thickness. The adequate thickness of a double-polymer membrane depends on the permeability of the conductive polymer layer. A thinner permeable layer provides a lower detection limit as long as the film is thick enough to retain the flow effectively. Components within the aqueous phase seriously limit the lifetime of the thinner membrane. The lifetime of the sensor is determined by repeating the measurement until the loss of membrane components deteriorates the detection limit.

Without wishing to be bound by theory: Using the solid-supported SP-ISE, we will establish ion transfer voltammetric of sodium ion analysis. We can develop PEDOT: PSS/GO as a solid support on SC-SP-ISE. We can also apply antifouling materials, and the sensor's selectivity will be studied with artificial tears. The repeatability of the sensor membrane will be evaluated in each composition for at least five ISEs.

The biosensor morphology can be investigated at each step using scanning electron microscopy (SEM).

Selective ion stripping voltammetry can enable the analysis of sodium in tears via a cheap and disposable sensor.

The artificial tear sample can evaluate the osmolarity level (FIG. 10). Using paper electrodes to reduce the size of the two-polymer system will help make a commercial probe.

Without wishing to be bound by theory, the sensor membrane can be covered in antifouling compositions to maximize sensitivity and selectivity and minimize fouling. The sodium ion can be detected in artificial and actual tear samples. Non-limiting, new aspects of the disclosure are the affordable and disposable screen-printed electrodes along with PEDOT/PSS/GO-coated electrodes. This composition can increase the charge delivery capacities and decreases the impedance relative to the bare electrodes. The SC-SP-ISE can be incorporated into a single probe.

### Example 4

### Example 4: Non-Limiting, Exemplary Screen-Printed Electrode (SPE) Protocol

In embodiments, the screen-printed electrode can be prepared as follows:

A carbon screen-printed electrode (SPE) can be coated first with a conductive polymer and then with a PVC membrane (ion-sensing membrane) involves the following steps:

Materials: Carbon screen-printed electrode (SPE), Conductive polymer (e.g., PEDOT:PSS or polyaniline), PVC powder, Plasticizer (e.g., dibutyl phthalate, DBP), Dissolving solvent (e.g., tetrahydrofuran, THF), and Electrolyte (optional, depending on application)

### Non-Limiting, Exemplary Procedure:

(1) Cleaning the Carbon SPE: Clean the surface of the carbon screen-printed electrode using a mild detergent solution followed by rinsing with deionized water. Dry the electrode thoroughly using nitrogen gas or under a stream of air. (2) Conductive Polymer Coating: Prepare a solution of the conductive polymer (e.g., PEDOT:PSS) onto the carbon SPE by drop casting or Electropolymerization. (3) PVC Membrane Preparation: Prepare a PVC membrane by dissolving PVC powder in a solvent like tetrahydrofuran (THF) to create a homogeneous solution. Add a plasticizer to the solution to increase the flexibility and stability of the PVC membrane. Add Na⁺ ionophore. Mix the components thoroughly to ensure an even consistency. (4) Coating the PVC Membrane: Apply the PVC solution onto the conductive polymer coated SPE surface using: Drop casting: Drop a small amount of the solution on the electrode and allow it to spread evenly, then let it dry. After the PVC layer is applied, allow the electrode to dry completely in a clean, dust-free environment. (5) Inspection: Inspect the final electrode for uniformity in the conductive polymer and PVC membrane coatings. Check the adhesion of the layers to ensure they are firmly bonded to the carbon surface. This procedure will yield a carbon SPE with a conductive polymer layer followed by a PVC membrane.

### Example 5

Example 5: Non-Limiting, Exemplary Fabrication of a Microsensor Comprising 3 Electrodes (see, e.g., FIG. 6)

Working Electrode (Carbon Fiber): The working electrode can be made of carbon fiber, or another material with high conductivity, electrochemical stability, and small size. Carbon fibers can be fabricated with very fine diameters, enabling precise control over the electrochemical measurement. These fibers are often used for recording currents in electrochemical experiments because they provide a good balance of stability and sensitivity. In this case, the fiber is integrated into a micro glass pipet, giving the electrode a compact and robust design for small-scale applications. Micro Glass Pipet: The entire electrode assembly can be housed in a micro glass pipet. In non-limiting, exemplary embodiments, the tip can have a diameter between about 2-4 mm. This glass pipet can position the electrodes in the solution and provide mechanical stability while also serving as a micro-channel for the analyte solution. The small diameter of the tip helps to confine the measurement to a localized area, which can lead to high-resolution sensing. Disposable Tip for Solution Sucking: The electrode assembly can be equipped with a disposable tip that allows for the aspiration of solution. This can facilitates sampling and removing small volumes of solution without contamination, which is important for maintaining controlled experimental conditions, especially when working with biofluids or other sensitive materials. Fabrication of Carbon Fiber Electrodes Using a Pipet Puller: In embodiments, the carbon fiber electrode can be created using a pipet puller. The pipet puller allows the creation of highly tapered, fine glass tips that can be further integrated with the carbon fiber and other components to create a precise microelectrode suitable for electrochemical measurements. The materials and design-carbon fiber for the working electrode, Ag/AgCl for the reference, platinum for the counter, and a micro glass pipet for housing—are chosen for their electrochemical properties, ease of fabrication, and durability in small-scale applications.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A screen-printed, solid contact ion-sensitive electrode (SP-SC-ISE), comprising one or more screen-printed electrodes, wherein at least one electrode is coated with a sensing membrane comprises a conducting layer and an ion-sensing membrane.

2. The SP-SC-ISE of claim 1, where in the screen-printed electrode is selected from a carbon electrode or a glass carbon electrode.

3. The SP-SC-ISE of claim 1, wherein the conducting layer comprises one or more conductive polymers, nanoparticles, or composite thereof.

4. The SP-SC-ISE of claim 3, wherein the conductive polymer or composite thereof comprises poly (3,4-ethylene dioxythiophene) polystyrene sulfonate (PEDOT:PSS), graphene oxide (GO), PTh Polythiophene (PTh), and Polyacetylene (PA), or a combination thereof.

5. The SP-SC-ISE of claim 1, wherein the ion-sensing composite membrane comprises a polymer, a solvent mediator, an ionophore, and a solvent.

6. The SP-SC-ISE of claim 5 wherein the polymer comprises polyvinyl chloride (PVC).

7. The SP-SC-ISE of claim 5, wherein the ionophore comprises a Na⁺ ionophore.

8. The SP-SC-ISE of claim 7, wherein the Na⁺ ionophore comprises lipophilic sodium tetrakis (4-fluorophenyl) borate dehydrate Na-ionophore.

9. The SP-SC-ISE of claim 8, wherein the solvent comprises tetrahydrofuran (THF).

10. The SP-SC-ISE of claim 8, wherein the solvent mediator is selected from the group consisting of 2-nitrophenyl octyl ether (O-NPOE), Dioctylsebacate (DOS), di-2-ethylhexyl phthalate (DEHP), or any combination thereof.

11. The SP-SC-ISE of claim 1, wherein the ion-sensing composite membrane has less than about 10% cross-sensitivity to contaminants.

12. The SP-SC-ISE of claim 1, wherein the one or more screen-printed electrodes comprise a working electrode, a reference electrode, and a counter electrode.

13. The SP-SC-ISE of claim 12, wherein the conducting layer deposited on the surface of the working electrode or on the surface of the working electrode, the reference electrode, and the counter electrode.

14. The SP-SC-ISE of claim 1, wherein the ion-sensing composite membrane is deposited on the conducting layer.

15. The SP-SC-ISE of claim 1, wherein the SP-SC-ISE is disposable.

16. The SP-SC-ISE of claim 1, wherein the SP-SC-ISE is paper-based.

17. An electrochemical sensor for measuring the osmolarity of a sample comprising one or more electrodes and a sensing membrane, wherein the sensing membrane comprises a conducting layer and an ion-sensing membrane.

18. The sensor of claim 17, wherein the one or more electrodes comprise a screen-printed electrode (SPE).

19. The sensor of claim 17, wherein the sensor is a voltametric sensor.

20. The sensor of claim 17, wherein the sensor is a microsensor.

21. The sensor of claim 17, wherein the sensor is disposable.

22. The sensor of claim 17, wherein the sensor is reusable.

23. The sensor of claim 20, wherein the microsensor comprises disposable tips.

24. The sensor of claim 17, wherein the sensor is paper based.

25. The sensor of claim 17, wherein the one or more electrodes is selected from a counter electrode, a working electrode, a reference electrode, or a combination thereof.

26. The sensor of claim 25, wherein the counter electrode comprises platinum.

27. The sensor of claim 25, wherein the working electrode is selected from a glass carbon electrode or a carbon electrode.

28. The sensor of claim 25, wherein the reference electrode comprises silver/silver chloride (Ag/AgCl) or saturated calomel electrode (SCE).

29. The sensor of claim 25, wherein the working electrode is coated with the sensing membrane of claim 16.

30. The sensor of claim 25, wherein the sensing membrane contacts the working electrode, the counter electrode, and the reference electrode.

31. The sensor of claim 17, wherein the sensor can perform ion-transfer stripping voltammetry (ITSV) or can be subject to ion-transfer stripping voltammetry.

32. The sensor of claim 17, wherein the conductive layer comprises one or more conductive polymers, nanoparticles, or composite thereof.

33. The sensor of claim 32, wherein the conductive polymer or composite thereof comprises poly (3,4-ethylene dioxythiophene) polystyrene sulfonate (PEDOT:PSS), graphene oxide (GO), PTh Polythiophene (PTh), and Polyacetylene (PA), or a combination thereof.

34. The sensor of claim 17, wherein the ion-sensing membrane comprises an ionophore composite.

35. The sensor of claim 34, wherein the ionophore composite comprises a polymer, a solvent mediator, an ionophore, and a solvent.

36. The sensor of claim 35, wherein the ionophore comprise a Na⁺ ionophore.

37. The sensor of claim 36, wherein the Na⁺ ionophore comprises lipophilic sodium tetrakis (4-fluorophenyl) borate dehydrate Na-ionophore.

38. The sensor of claim 35, wherein the polymer comprises polyvinyl chloride (PVC).

39. The sensor of claim 35, wherein the solvent comprises THF.

40. The sensor of claim 35, wherein the solvent mediator is selected from the group consisting of 2-nitrophenyl octyl ether (O-NPOE), Dioctylsebacate (DOS), di-2-ethylhexyl phthalate (DEHP), or any combination thereof.

41. The sensor of claim 17, wherein the sensor has a detection limit of about 0.005 mM.

42. The sensor of claim 17, wherein the sensor can measure a sample volume of about 1 μL to about 50 μL.

43. The sensor of claim 17, wherein the sensor further comprises an anti-fouling agent.

44. The sensor of claim 43, wherein the anti-fouling agent comprises a polar chemical group, hydrophilic polymers, self-assembled layers, or a combination thereof.

45. The sensor of claim 17, wherein the sensor comprises a microfluidic suction probe or flow-through sensor.

46. A method of diagnosing Dry Eye Disease (DED), the method comprising:
applying the sensor of claim 17 to the tears of a subject;
measuring the osmolarity of the tears; and
determining if the subject is afflicted with Dry Eye Disease (DED) based upon the osmolarity.

47. The method of claim 46, wherein the measuring osmolarity comprises measuring the physical or electrochemical signal of a target analyte in the sample, converting the sensor signal to osmolarity by measuring the sensor signal at known concentrations of a reference solution to generate a concentration curve, and multiplying the concentration of the sodium ion by the van't Hoff factor, 2.
